SEQUENCE LISTING (14 sequences)

SEQ ID NO: 1 - DNA, 48 bp, Artificial Sequence
  synthetic primer
gatcttaattaactcataggcatcatgcatctcctcaacgggaaaacg

SEQ ID NO: 2 - DNA, 41 bp, Artificial Sequence
  synthetic primer
gatcggcgcgccctaataattatttactcgaagtgaatccc

SEQ ID NO: 3 - DNA, 1974 bp, Fusarium graminearum
atgcatctcctcaacgggaaaacggcagcggttgcgttaggtgcataaagaccagcacctattctacagctaatgctaacaatctttacagcgcttctcaattcgtgcaatgctctcaaggtcaccatccttgcagacaccaatcgcgatggcaaggtcgacaacaacgacattaatggcaaatccacgtggacaaacaacagaggcgctttgattcttcccaacattggcgataccggaagccgttgtgctaagcaatggggaccaagtgtcgacatccaaggagatgaatcttatttggacaaatgtaacgacgcctcagacaatgtgcagcgaaatcctaaatatctcgcttcacttaagactctcccattgactaccttgagtgcaacggccaaaggttcaatcatcattgccgacaaaacaggcgcctccaaagttcgaatctttgtaaagcagagcggcaagtggaactacgtggctgcagaccatgtcttcactgccaaggagctcaaatccggactcgagctgggtgtcgatgcccgtgatgtccgtcgtcctcaagattggaatggttacgccaagatccagttcactgtcaccgatggcaagaccaaagcgactgacgccgttgctgtacgagttgcgcctgtgctgacgcatcatcatggacaacacgctcagcgcatcttcacaaccggtgtcaatgaagcaggtgtcaacaaagtccaagagacattcattgctgatattctacgcaatgtggctggcgctggcatcaaagagcctgtgttccagttccataaccaggatatctggacgcaggacttttttgagcctggttacgccagcattcctggccccaatggacccgtcagtatccgtatcatgattcgatcggcccagagcagtcgaagatctggtcgtgacgcattccacgaccttcgcaatgatcaagtcggtgctgttcagcatcctggcgatggagactcgatcgactccactggtaacctggagaccattcctccttacagccacaacggaaagagtttccccgtcggcagaaccatcatgggtgcatgggacggaagagctcctctcatggtcgaattcctcaaagctcaacaggtccaggaacctttgattctcgacacaagctggctttacgtgggccacgtggatgaattcattcagttcctacccagcaataacaagctcggctgggtcatcatggttgctgatccgatgaagggcgtcgatctcttgaagaaggcggtcaagaccggtcatggcaaagtaaaagctgtgtcacgtcctctgtcggcagatgagaagaaagaacaattgtgtcttccccgacagactattgctgaggcgcttaagtttaaaagttttgatgccatcaacaagcattcggctgagcgcattcaagcaaatcttgatattatcaagcgagagactggcatcacagatgaagacatccatcgtgttcctgcacttttctactacacccagtcgaacagttggctatgtcccggcgagactgcagaggatgattcagctcagcctcaaaaggctgcgagcaactcgggtatcacaatgaagacttcccagggtggtcccggcttcaaggccaagtccatcgtcgaagcagccactccaggaaagtctattcagcgtcgagtgatcgaccccgctactcaggtcacggctctttaccctggtagcgtcaatggtctggtcatgacagataccaagattcttgcgcctagcccttggggtcccgtgatcaacaagcaggacatctttgctgctgctgtatcccaagtatacacgaacgccggatacaatgtcacataccaggatgattggttctctcactttaagttgcaaggagatgttcactgcggtagcaattcgtggagggaaattccaaagaagtggtgggattcacttcgagtaaataattattag

SEQ ID NO: 4 - DNA, 1974 bp, Fusarium graminearum
atgcatctcctcaacgggaaaacggcagcggttgcgttaggtgcataaagaccagcacctattctacagctaatgctaacaatctttacagcgcttctcaattcgtgcaatgctctcaaggtcaccatccttgcagacaccaatcgcgatggcaaggtcgacaacaacgacattaatggcaaatccacgtggacaaacgacaaaggcgctttgattcttcccaacattggcgataccggaagccgttgtgctaagcaatggggaccaagtgtcgacatccaaggagatgaatcttatttggacaagtgtaacgacgcctcagacaacgtgcagcgaaatcccaaatatctcgcttcacttaagactctcccattgaccaccttgagtgcaacggccaaaggttcaatcaccattgccgacaaaacaggcgcctccaaagtccgaatctttgtcaagcagagcggcaagtggaattatgtggctgcagaccatgtcttcacagccaaggagctcaaatccggactcgagctgggtgtcgatgcccgtgatgtccgtcgtcctcaaggttggaatggttacgccaagatccagttcactgtcaccgatggcaagaccaaagcgactgacgccgttgctgtacgagttgcgcctgtgctgacgcatcatcatggacaacacgctcagcgcatcttcacaaccggtgtcaatgaagcaggtttcaacaaagtccaagagaaattcattgctgatgttctacgcaatgtggctggcgctggcatcaaagagcctgtgttccagttccacaaccaggatatctggacgcaggacttttttgagcctggttacgccagcattcctggccccaatggacccgtcagtatccgtatcatgattcgatcggcccagagcagtcgaagatctggtcgtgacgcattccacgaccttcgcaatgatcaagtcggtgctgttcagcatcctggcgatggagactcgatcgactccactggtaacctggagaccattcctccttacagccacaacggaaagagtttccccgtcggcagaaccatcatgggtgcatgggacggaagagctcctctcatggtcgaattcctcaaagctcaacaggtccaggagcctttgattctcgacacaagctggctttacgtgggccacgtggatgaattcattcagttcctacccagcaataacaagctcggctgggtcatcatggttgctgatccgatgaagggtgtcgatctcttgaagaaggcggtcaagaccggtcatggcaaagtaaaagctgtgtcgcgtcctctgtcggcagatgagaagaaagaacaattgtgtcttccccgacagactattgctgaggcgcttaagtttaaaagttttgatgccatcaacaagcattcggctgagcgcattcaagcaaatcttgatattatcaagcgagagactggcatcacagatgaagacatccatcgtgttcctgcacttttctactacacccagtcgaacagttggctatgtcccggcgagactgcagaggatgattcagctcagcctcaaaaggctgcgagcaactcgggtatcacaatgaagacttcccagggtggtcccggcttcaaggccaagtccatcgtcgaagcagccactccaggaaagtctattcagcgtcgagtgatcgaccccgctactcaggtcacggctctttaccctggtagcgtcaatggtctggtcatgacagataccaagattcttgcgcctagcccttggggtcccgtgatcaacaagcaggacatctttgctgctgctgtatcccaagtatacacgaacgccggatacaatgtcacataccaggatgattggttctctcactttaagttgcaaggagatgttcactgcggtagcaattcgtggagggaaattccaaagaagtggtgggattcacttcgagtaaataattattag

SEQ ID NO: 5 - DNA, 1923 bp, Fusarium graminearum
atgcatctcctcaacgggaaaacggcagcggttgcgttagcgcttctcaattcgtgcaatgctctcaaggtcaccatccttgcagacaccaatcgcgatggcaaggtcgacaacaacgacattaatggcaaatccacgtggacaaacaacagaggcgctttgattcttcccaacattggcgataccggaagccgttgtgctaagcaatggggaccaagtgtcgacatccaaggagatgaatcttatttggacaaatgtaacgacgcctcagacaatgtgcagcgaaatcctaaatatctcgcttcacttaagactctcccattgactaccttgagtgcaacggccaaaggttcaatcatcattgccgacaaaacaggcgcctccaaagttcgaatctttgtaaagcagagcggcaagtggaactacgtggctgcagaccatgtcttcactgccaaggagctcaaatccggactcgagctgggtgtcgatgcccgtgatgtccgtcgtcctcaagattggaatggttacgccaagatccagttcactgtcaccgatggcaagaccaaagcgactgacgccgttgctgtacgagttgcgcctgtgctgacgcatcatcatggacaacacgctcagcgcatcttcacaaccggtgtcaatgaagcaggtgtcaacaaagtccaagagacattcattgctgatattctacgcaatgtggctggcgctggcatcaaagagcctgtgttccagttccataaccaggatatctggacgcaggacttttttgagcctggttacgccagcattcctggccccaatggacccgtcagtatccgtatcatgattcgatcggcccagagcagtcgaagatctggtcgtgacgcattccacgaccttcgcaatgatcaagtcggtgctgttcagcatcctggcgatggagactcgatcgactccactggtaacctggagaccattcctccttacagccacaacggaaagagtttccccgtcggcagaaccatcatgggtgcatgggacggaagagctcctctcatggtcgaattcctcaaagctcaacaggtccaggaacctttgattctcgacacaagctggctttacgtgggccacgtggatgaattcattcagttcctacccagcaataacaagctcggctgggtcatcatggttgctgatccgatgaagggcgtcgatctcttgaagaaggcggtcaagaccggtcatggcaaagtaaaagctgtgtcacgtcctctgtcggcagatgagaagaaagaacaattgtgtcttccccgacagactattgctgaggcgcttaagtttaaaagttttgatgccatcaacaagcattcggctgagcgcattcaagcaaatcttgatattatcaagcgagagactggcatcacagatgaagacatccatcgtgttcctgcacttttctactacacccagtcgaacagttggctatgtcccggcgagactgcagaggatgattcagctcagcctcaaaaggctgcgagcaactcgggtatcacaatgaagacttcccagggtggtcccggcttcaaggccaagtccatcgtcgaagcagccactccaggaaagtctattcagcgtcgagtgatcgaccccgctactcaggtcacggctctttaccctggtagcgtcaatggtctggtcatgacagataccaagattcttgcgcctagcccttggggtcccgtgatcaacaagcaggacatctttgctgctgctgtatcccaagtatacacgaacgccggatacaatgtcacataccaggatgattggttctctcactttaagttgcaaggagatgttcactgcggtagcaattcgtggagggaaattccaaagaagtggtgggattcacttcgagtaaataattattag

SEQ ID NO: 6 - protein, 640 aa, Fusarium graminearum
MHLLNGKTAAVALALLNSCNALKVTILADTNRDGKVDNNDINGKSTWTNNRGALILPNIGDTGSRCAKQWGPSVDIQGDESYLDKCNDASDNVQRNPKYLASLKTLPLTTLSATAKGSIIIADKTGASKVRIFVKQSGKWNYVAADHVFTAKELKSGLELGVDARDVRRPQDWNGYAKIQFTVTDGKTKATDAVAVRVAPVLTHHHGQHAQRIFTTGVNEAGVNKVQETFIADILRNVAGAGIKEPVFQFHNQDIWTQDFFEPGYASIPGPNGPVSIRIMIRSAQSSRRSGRDAFHDLRNDQVGAVQHPGDGDSIDSTGNLETIPPYSHNGKSFPVGRTIMGAWDGRAPLMVEFLKAQQVQEPLILDTSWLYVGHVDEFIQFLPSNNKLGWVIMVADPMKGVDLLKKAVKTGHGKVKAVSRPLSADEKKEQLCLPRQTIAEALKFKSFDAINKHSAERIQANLDIIKRETGITDEDIHRVPALFYYTQSNSWLCPGETAEDDSAQPQKAASNSGITMKTSQGGPGFKAKSIVEAATPGKSIQRRVIDPATQVTALYPGSVNGLVMTDTKILAPSPWGPVINKQDIFAAAVSQVYTNAGYNVTYQDDWFSHFKLQGDVHCGSNSWREIPKKWWDSLRVNNY

SEQ ID NO: 7 - protein, 640 aa, Fusarium graminearum
MHLLNGKTAAVALALLNSCNALKVTILADTNRDGKVDNNDINGKSTWTNDKGALILPNIGDTGSRCAKQWGPSVDIQGDESYLDKCNDASDNVQRNPKYLASLKTLPLTTLSATAKGSITIADKTGASKVRIFVKQSGKWNYVAADHVFTAKELKSGLELGVDARDVRRPQGWNGYAKIQFTVTDGKTKATDAVAVRVAPVLTHHHGQHAQRIFTTGVNEAGFNKVQEKFIADVLRNVAGAGIKEPVFQFHNQDIWTQDFFEPGYASIPGPNGPVSIRIMIRSAQSSRRSGRDAFHDLRNDQVGAVQHPGDGDSIDSTGNLETIPPYSHNGKSFPVGRTIMGAWDGRAPLMVEFLKAQQVQEPLILDTSWLYVGHVDEFIQFLPSNNKLGWVIMVADPMKGVDLLKKAVKTGHGKVKAVSRPLSADEKKEQLCLPRQTIAEALKFKSFDAINKHSAERIQANLDIIKRETGITDEDIHRVPALFYYTQSNSWLCPGETAEDDSAQPQKAASNSGITMKTSQGGPGFKAKSIVEAATPGKSIQRRVIDPATQVTALYPGSVNGLVMTDTKILAPSPWGPVINKQDIFAAAVSQVYTNAGYNVTYQDDWFSHFKLQGDVHCGSNSWREIPKKWWDSLRVNNY

SEQ ID NO: 8 - protein, 623 aa, Chaetomium globosum
MAISFRRLLSLVPLGLVPSAAATFRPRILADTNRDGVVNHLDEPHKHDWTASRGAIFLPNIGDSTHRCPAVDLTGAPLSNDELWRCHDASGGRLLPSSAKYAAPLLTVPLPDVSDAAVGRIYTEPAHTRDRVRLFWKENDWSTGYSDVWATVDRQVTFNATSLRKGITLAMDGRELVTDAEVWDGSVKVVFEVTDGGRTARDFVAMKQAPVLLHHHLQRAEVVLSTAAGDNKTIGSSWQAHFLRGLEKAIDGDVPIVLFNQSTDIWAQDFLEPGYASMPGPDGPISIRILLRSAQSTREAGRQVFSQLRSAGIGGFQPGSGSGFGWEEINSGGNIETIPPYTSRTGKRWPNGRVIMGQHFGTYPAESMVKFLQSQQAQTPLFLETGWLAVGHVDEMVQFLPTTTTPLNFTLAIADTTAALTLLQHANASGHGSTPFVSYAGDATPDELTLFLDPTLLHNTTTIASILSSPSFLTTQAHAQHHLDATLTTLLRELPLRETDILRVPTLFHDVTYPWPTTPDGHPSRLARAPARERQLKGLVPQAINGLVVGGGGSGKYVAPRQWGPVVGGRDVFGEAVEEVYGRVGMRVVWVDDYMSHHVRGGEVHCGTNALREMGSWWEGSGE

SEQ ID NO: 9 - protein, 654 aa, Streptomyces scabies
VRSRIRRRIHPAGPMVLALAAAGSVLAGPALPASASGPATRADLRADVNRDGRIDVSGDTDTSGEDGWSVSRGAVHLPNLDDDTKRCPATGPGGKPLSDAKLAACNDASDTKVNGSADAADLARIRSLPLPGLPADAEGSLKVTAGTKHTHVFLKRSGTWTLVTSRTRLTAAELRSGVEFGVEATDVIRDSARWDGRAVIRLTVKSGEGTSSDSVTLRTAPLLTQHHLQDTQQVMVTKVRGEGEYGRLQRKFVKDLAREVKAAGVTAPLVTFTKYGDPWAQDFVEPAYVSMTGPGGRPQVMRVMLRSAQLDRESGRELFERIRGRDIGVVQVTDPVPPADWSLNSMGNLETVPPYAHNGKSYPAGRIIMGERKGSDIRPSKVMRTMLTSQGLQNPLLLDTSWLGVGHVDEFVQFLPADTPRGWRIGLADPSAGLKLLRDAERAGHGSTKVFSVPRTKGAPAPKETIAQALASRHLVADNEMAAERIAANLEILKRETGVTDGEIVRVPALYTRDVMEQAADGQSVPVPRLTRMGGGSALVNSLKEHGQQKWLADNPAASRAKAPAPVMTSAYVPGAVNGVLLGRDRYLAPRQWGPVIGGKDIFTEAVTAVYTRAGLKVSYIDDWYTYHLGQGEVHCGTNTLRDVSTAWWAGSAR

SEQ ID NO: 10 - protein, 583 aa, Streptomyces scapies
VPTRSTRPLRTRSARQAIAVLAVSGTLLATPSLAAPSAFAAGAPPTDLRVDTNRDGNVDVTGTTDTAGENGWTVARGALMLPNIDDDTKRCPVTGPKGKPLSDAKLAACNDASDTKVNGTADAADLARVRSVPMAGVPAGAQGTVKVTAGAAQTRVFVKRGTKWEPVTAKTRLSRAELKAGVEFGVEARDVIRDTAKWDGTARVRLTVTSSKGTTADSVTLRVAPLLTHHHLQNAQQMLVTKISGSDHGGYAKLNRVFREGLDKAAKSAGITKPTVNFTKYGDIWAQDFVEPAYVSMTGADGKRQAMRVMLRSAQLDREAGRELFEKMRGPNIGAVQVTGRKDSEEWTLNSMGNLETIPPYAQGDRSYPAGRIIMGQRPDTGSKPAKVMRTFLKSQGLQDPLFLDTSWLHVGHVDEFVQFLPADTPRGWKIAIADPAAGLKLLRDAKAAGHGSTSMFSLPGVPWESISEALGSKHLVSDNNLATRRIEENLAVLQRETGVTDAEVVRVPALYTRGREMVGESQSGGDEKLRLPRLTRLGAGAELPEAARDYGQQRRLDDRAADQGALTPDSSAAASPAAPAPT

SEQ ID NO: 11 - protein, 9 aa, Artificial Sequence
  consensus sequence
WLXVGHVDE

SEQ ID NO: 12 - protein, 11 aa, Artificial Sequence
  synthetic peptide
QPRPFPFPRPR

SEQ ID NO: 13 - protein, 558 aa, Streptomyces clavuligerus
VRVTGWRNAAVATGVAGALIGTAGVVHAVGPAAGPAARAALTADLRADVDRDGSVDITGGSDTPGENVWTRDRGAVMLPNIDDDTKRCPVRTAQGKPLTDAELAKCNDAADTVLNGASDAADLARLKTVPLPEVSDGAYGTAVVLAKDRARLFVKRANGWTHLRPTDRLTAAELRAGVELGIEATDVIRNATVWDGQAIVRFSVTDSTTTVKDDVRMGTAPVLTHHHRQKAQEVLVTAVKGEKLQEKFVKDLEREVKAAGITKPVRKLGGAGGDRWAQDFLEPGYVSMTGPGGKPHAMRVMIRSAQDRKAGREVFEKLRGKDIGAVQMSGRLGWNTLDSMGNLETIPPYAHGGKEYPAGRIIMGHQPDARNRPAKSMSTFLRSQGAQSPLLLDTAWLGVGHVDEFVQFLPAATERGWRIGVADPQAGVALLRKAQKDGHGRAKMFSVPNGPEGQAPKQTIDEVLKNRSFHADNKLATERIEANLRILKNETGITDAEIIRIPGLYSRGDMDGSVFGGGRAKLSRLDIDTALTFDQAGPDRKQPFAASGRAGERAWPTG

SEQ ID NO: 14 - protein, 606 aa, Phaeosphaeria nodorum
MKPASCLLFSGFAFQAASAATWSPDLRADTNRDGIVDVVGSSDRSNKTAWNAELEAIFLPNIGDKYLRCANTDKVGNPLSNDELAACNDASGHLLLAPEYVAPLRTVPMNVSDDVAARIYATPQAAYERVRLFVLDNPEDKNSTTSWRLVDQEFQFNATQLRAGITLGIDGRELVTNASVWDGSVVIRFDVFNDLDQSSDSVAMKMAPVLIHHHLQKVETIIATDSNSTNPDQQNFVRQIEAARQTAGIANQLILFNQSDDIWAQDFIEPGYASMPGPNGPIAIRVILRSAQSTRTGGRQVFEQLRGPGVGGFQPSAGTGSGFGHREINSFGNLETIPPYTSKSGESYPVGRIIMGKHFEEKPAAAVLDFLHDQQLQSPLLLETGWLLIGHVDEFVQFLPYDNELGFTIAIADTVSGMNMLRKLQTDGHGGIRAISFPGDTGASVPDKNGTINQLLSNATFVEANNYAQRHIDANLQTLLAEIPLMAEDIIYVPTLFQESNFGSGFGSPDGLPSRFATIPANEKLLVAFSPAAINGIVIGSQYFSPKPWGPVVNGSDVLERAVEAAYARAGMSVGFVDDFLSHHVGGGEIHCGSNTLRQTDAIWWE

(19) United States
(12) Patent Application Publication
(10) Pub. No.: US 2009/0297689 A1
(43) Pub. Date: Dec. 3, 2009

(54) PEPTIDYLARGININE DEIMINASE AND USES THEREOF IN THE PRODUCTION OF CITRULLINATED PROTEINS AND PEPTIDES

(76) Inventors: Luppo Edens, Rotterdam (NL); Nicolass Emile Paulus Deutz, Little Rock, AR (US); Petrus Jacobus Theodorus Dekker, Den Haag (NL)

Correspondence Address: NIXON & VANDERHYE, PC, 901 NORTH GLEBE ROAD, 11TH FLOOR, ARLINGTON, VA, 22203, US

(21) Appl. No.: 12/306,469

(22) PCT Filed: Jun. 25, 2007
(86) PCT No.: PCT/EP2007/056310

§ 371 Date: Jun. 26, 2009

(30) Foreign Application Priority Data
Jun. 26, 2006 (EP) 06116084.2
Sep. 7, 2006 (EP) 06120272.7

(51) Int. Cl. A23J 1/00; C12P 21/06; C12N 9/14; C12N 5/10; C12N 15/63; C07K 5/08; C07K 7/06; C07K 5/10; C07K 14/00; C07H 21/04

(52) U.S. Cl. 426/656; 435/68.1; 435/69.1; 435/195; 435/325; 435/320.1; 530/300; 530/324; 530/325; 530/326; 530/327; 530/328; 530/329; 530/330; 530/331; 530/350; 536/23.2

(57) ABSTRACT

The present invention relates to a protein, peptide or protein hydrolysate wherein the molar ratio of citrulline and arginine residues, being part of protein or peptide, is at least 0.15, preferably at least 0.30, more preferably at least 0.5, still more preferably at least 1.0, even still more preferably 2.0 and most preferably at least 4.

### FIELD OF THE INVENTION

The present invention relates to proteins and peptides comprising citrulline residues.

### BACKGROUND OF THE INVENTION

Arginine is a conditionally essential amino acid playing a key role in mammalian physiology. The metabolic pathways of arginine have been well described. Upon its dietary intake, arginine is taken up from the hepatic portal vein by the liver and rapidly converted into ornithine by the enzyme arginase. In the latter process, urea is formed. The ornithine generated from arginine is then converted into citrulline, or can be metabolized to the amino acids glutamate and proline. Alternatively the ornithine formed is incorporated into polyamine compounds such as putrescine. Dietary arginine that is not metabolized to ornithine, can be processed to a.o. nitric oxide or to arginyl-tRNA for the purpose of protein synthesis. Also an endogenous synthesis route towards arginine exists. The latter process takes place primarily in the kidney where arginine is synthesized from ornithine and citrulline precursors.

Citrulline is a natural amino acid that has been described to occur as a free amino acid in cucurbitaceous fruits like watermelons, pumpkins and cucumber. Other sources of the free amino acid are prune juice, some grape variants and in fermented foods such as soy sauce and wines. In nature citrulline rarely occurs linked to other amino acids. In edible mushrooms the presence of the dipeptide pyroglutamate-citrulline has been shown and in Irish moss the dipeptide citrulline-arginine. In mammals the presence of low levels of peptides or proteins incorporating citrulline has been shown using immunochemical techniques.

In mammals citrulline is synthesized in the gut from glutamine, released into the blood and converted back into arginine in the kidneys. In healthy adults, the citrulline converted by the kidney is enough to provide the body's full arginine requirements. However, in newborns, this citrulline to arginine reaction in the kidneys is inadequate and additional mechanisms are involved. The important role of citrulline as an alternative for arginine in various physiological processes is being elucidated by recent research. Because the capture of dietary arginine by the liver is so efficient, arginine concentrations in the blood downstream of the liver are relatively low. So conditions may arise, e.g. during periods of rapid growth, as the result of malnutrition or changes in the amino acid metabolism, or in response to traumatic or pathologic insults, where the demand for arginine in all organs may not be fully met. In such situations citrulline may act as an alternative to arginine. In contrast to dietary arginine, dietary citrulline is not withdrawn from the portal blood by the liver. So, citrulline represents an alternative, but more efficient, source of freely circulating arginine available to peripheral tissues including muscles (Curis at al., Amino Acids (2005) 29:177-205). Dietary citrulline also does not lead to ureagenesis in the liver as has been described for the arginase reaction in which arginine is converted into ornithine. Therefore, negative side effects of supplemental arginine such kidney damage as the result of this ureagenesis and a decrease of immunological status, can be avoided by taking citrulline. Yet another advantage of citrulline is that it can trap excess ammonia, i.e. it can act as a so-called hypoammonaemic agent offering advantages for patients suffering from certain enzymic dysfunctions, individuals suffering from epilepsy and, for healthy individuals, for preventing fatigue resulting from prolonged, high intensity muscular efforts.

In many countries regulations are in place that legislate against the addition of free amino acids to food. As a consequence, free amino acids can only be used in clinical nutrition so that the above-mentioned physiological advantages of supplemental arginine or citrulline cannot be made available to consumers at large. Furthermore the very bitter taste of free arginine forms an important drawback for clinical and non-clinical applications. The critically ill will simply refrain from food or supplements with a bad taste and so will consumers with non-medical needs, such as elderly or sports people. Thus, the use of free amino acids in foods can be expected to cause serious palatability problems, certainly if the recommended amino acid dosages are taken into account. The implication of these conclusions is that there exists a clear need for citrulline in a form in which it is not present as a free amino acid and in which it has an improved palatability.

### SUMMARY OF THE INVENTION

The present invention relates to a modified protein, peptide or protein hydrolysate wherein at least 15%, preferably at least 30%, more preferably at least 45%, still more preferably at least 60% and most preferably at least 80% of the arginine residues which are originally present in the protein, peptide or protein hydrolysate are transformed into citrulline residues. Therefore the protein, peptide or protein hydrolysate of the invention preferably has a molar ratio of citrulline to arginine (present in the protein, peptide or in protein hydrolysate) of at least 0.15, preferably at least 0.30, more preferably at least 0.5, still more preferably at least 1.0, even still more preferably 2.0 and most preferably at least 4. In case of a hydrolysate the amount of arginine present as free amino acid is not used in the determination of the citrulline formation, nor is taken into account of citrulline to arginine ratio. So the present invention relates to proteins, peptides or hydrolysates having a high ratio of bound citrulline residues. By bound citrulline or peptide bound citrulline is meant citrulline residue which is part of a peptide or protein, in contrast to free citrulline, which is a free amino acid.

Moreover the present invention relates to a method of enzymatically producing a protein, peptide or protein hydrolysate wherein at least 15%, preferably at least 30%, more preferably at least 45%, still more preferably at least 60% and most preferably at least 80% of the arginine residues which were originally present in the protein, peptide or protein hydrolysate are transformed into a citrulline residue. To obtain the protein, peptide or protein of the invention the starting protein, peptide or protein hydrolysate substrate is incubated with a protein arginine deiminase.

Furthermore it is an object of the present invention to provide a protein, a protein hydrolysate, a peptide or a mixture of peptides that can be used as a food, a food ingredient, a feed, a feed ingredient, nutraceutical, such as a dietary supplement or medicament, or an ingredient of a nutraceutical, such as a dietary supplement or medicament, or can be used as an ingredient in the production of a food, a feed or a nutraceutical, such as a dietary supplement or a medicament.
According to another aspect of the invention a protein arginine deiminase is disclosed which is actively secreted by a production host in the culture medium. The invention also relates to the production and use of such a protein arginine deiminase.

Therefore the present invention relates to an isolated polypeptide which has protein arginine deiminase activity, selected from the group consisting of:
(a) a polypeptide which has an amino acid sequence which has at least 30% amino acid sequence identity with amino acids 1 to 640 of SEQ ID NO: 6, 8, 9, 10, 13 or 14;
(b) a polypeptide which is encoded by a polynucleotide which hybridizes under low stringency conditions with (i) the nucleic acid sequence of SEQ ID NO: 3 or a fragment thereof which is at least 80% or 90% identical over 60, preferably over 100 nucleotides, more preferably at least 90% identical over 200 nucleotides, or (ii) a nucleic acid sequence complementary to the nucleic acid sequence of SEQ ID NO: 3.

### DETAILED DESCRIPTION OF THE INVENTION

Citrulline, while being an amino acid, is not coded for by DNA and is not built into proteins during protein synthesis. Yet, in several mammalian tissues minute quantities of peptide bound citrulline has been detected using immunochemical techniques. Examples are synovial fluid, synovial tissue, haematopoietic cells and activated macrophages. Proteins containing citrulline residues are generated in a so-called post-translational modification of peptide bound arginine residues. This particular modification is catalysed by a family of enzymes called protein or peptidyl arginine deiminases (EC 3.5.3.15), which convert peptide or protein bound arginine into peptide or protein bound citrulline in a process called citrullination or deimination. The term protein arginine deiminase and peptidyl argine deiminase are interchangeably used herein. In the reaction from arginine to citrulline, one of the terminal nitrogen atoms of the arginine sidechain is replaced by an oxygen. The reaction uses one water molecule and yields ammonia as a side product (http://en.wikipedia.org/wiki/Citrullination). Whereas arginine is positively charged at a neutral pH, citrulline is uncharged. Citrullination thus increases the hydrophobicity of the peptide or protein, a process that can alter the properties of proteins or peptide and may ultimately lead to protein unfolding. Mammalian proteins known to contain citrulline residues include myelin basic protein (MBP), filaggrin and several histone proteins, while other proteins, like fibrin and vimentin can get citrullinated during cell death and tissue inflammation. Note that citrullination of proteins is distinct from the formation of the free amino acid citrulline as part of the urea cycle or as a byproduct of enzymes of the nitric oxide synthase family.

In contrast to arginine containing proteins or peptides, citrullinated proteins or peptides are not commercially available on an industrial scale. Surprisingly we have identified a food grade and industrially applicable method comprising the production of protein or peptide bound citrulline. In this method use is made of the enzyme protein arginine deiminase or peptidyl arginine deiminase (hereinafter referred to as PAD) that efficiently converts protein or peptide bound arginine residues into protein or peptide bound citrulline residues.

Although several types of PAD's are known, predominantly from mammalian tissue, none of these enzymes is suitable for industrial application. By screening many microorganisms, we have quite unexpectedly come across some microorganisms that are able to actively secrete a PAD enzyme into the fermentation broth. It was not expected that such an actively secreted PAD could be found in nature since all mammalian-type PAD's that are currently described, are not actively secreted in the culture medium. Active secretion is defined here as the ability of an organism to accumulate a polypeptide in the growth or culture medium. Active secretion of a polypeptide requires energy from the host organism and a dedicated secretion pathway from the host organism. In general, polypeptides that are actively secreted contain an amino-terminal pre-sequence, also called signal sequences or signal peptide. Active secretion is not necessarily accompanied by the disruption of the cell wall to transport the enzyme to the fermentation broth. In general Gram-negative bacteria are known not to have active secretion.

Mammalian PAD's are strongly related to each other, and different isoforms are expressed in a variety of different organs. Indeed, the enzymatic PAD activity could only be detected after lysis of the cells, which strongly indicates that PAD is an intracellular enzyme in mammalian tissue (reviewed by Vossenaar et al (2003) Bioessays 25:1106-1118) and the enzyme is not actively secreted. Additionally; all mammalian PAD's lack a clear signal sequence that is normally required for efficient secretion. It seems therefore not logical to screen for actively secreted PAD enzymes in micro-organisms, although such enzymes would have a clear industrial advantage.

Active secretion is of paramount importance for an economical production process because it enables the recovery of the enzyme in an almost pure form without going through cumbersome purification processes. Overexpression of such an actively secreted PAD by a food grade fungal host such as Aspergillus, yields a food grade enzyme and a cost effective production process towards citrullinated proteins or peptides.

To the best of our knowledge, we are the first to describe a PAD that is efficiently secreted into the fermentation broth by a food grade host organism like Aspergillus and the first to report on a cost effective production process towards citrullinated proteins or peptides.

The enzyme arginine deiminase (EC 3.5.3.6) is well known and its use in the conversion of free arginine into free citrulline has been extensively described. However, PAD's (EC 3.5.3.15) form a relatively new family of enzymes that catalyse the deimination of protein or peptide bound arginine residues to produce protein or peptide bound citrulline residues hereby releasing ammonia. Sofar the enzyme has been identified in a large variety of mammalian tissues. In humans, for example, four different PAD enzymes have been identified that can be found in a.o. the skin, the uterus, muscles, brain, pancreas, spleen, stomach, thymus, spinal chord and in haematopoietic cells such as macrophages. These Ca2+-dependent PAD enzymes are involved in a.o. the differentiation of the epidermis, in the myelination of nerve axons and in the keratinization of hair follicles. None of the known mammalian PAD's are actively secreted by the cell. Although widely present in, mammalian tissues, reports on active PAD's from microorganisms are very limited. The single exception is a bacterial enzyme from the human Gram-negative pathogen Porphyromonas gingivalis (McGraw et al. (1999) Infect Immun. 67(7):3248-3256). This enzyme is transported to the periplasmatic space and only leaks from this periplasmatic space outside the bacterium. The initiation and progression of adult-onset periodontitis has been associated with infection of the gingival sulcus by Porphyromonas gingivalis. This Porphyromonas PAD is not evolutionarily related to the vertebrate PAD's but shares sequence homology with several arginine deiminases (Shirai et al. (2001) Trends Biochem Sci.; 26(8):465-468). However, the enzyme can convert peptide-bound as well as free L-arginine to citrulline and, in contrast to the mammalian PAD's, is not dependent on calcium ions. Therefore, the enzyme is not actively secreted, is highly unstable and has been described as virulence factor so food grade, industrial applications of this P. gingivalis derived enzyme are highly unlikely. Therefore PAD from Porphyromonas gingivalis and its use are not part of the present invention.

Here we have isolated and identified the secreted PAD from the fungus Fusarium graminearum that is to our knowledge the first secreted PAD that is described. On basis of a PAD assay we could isolate and characterize several variants of secreted PAD in Fusarium strains The genes encoding the PAD from these fungi were isolated and characterized (SEQ ID NO: 3 and 4). Overexpression of this PAD in the fungus Aspergillus niger leads to efficient secretion of the PAD into the culture medium. All these new variants contain the characteristics of a secreted PAD. Additionally, based on this knowledge, we could identify and correctly annotate the genes of secreted PAD's in other microorganisms of which the DNA sequences are present in public databases. Potentially secreted PAD's can be found in the fungi Chaetomium globosum, Phaeosphaeria nodorum and the bacteria Streptomyces scabies and Streptomyces clavuligerus. Based on our knowledge of the secreted character of these proteins, we suggest the correct protein sequences as depicted in SEQ ID NO: 8 to 10, 13, and 14. All these sequences have not been described as coding for a secreted peptidyl arginine deiminase before, but could only be identified as such because of our knowledge of the gene structure of the secreted PAD of Fusarium graminearum.

After an extensive screening of fungi from different culture collections, we have been able to identify Fusarium species as a source of a PAD-like enzyme that is actively secreted into the fermentation broth. This Fusarium derived PAD-like enzyme shows considerable homology with the mammalian PAD's, but not with the P. gingivalis PAD. Isolation of PAD from the Fusarium fermentation broth via chromatographic purification, revealed a molecular weight of this secreted PAD of approx 55 kDa, an activity optimum around pH 8 and a temperature optimum between 40 and 50 degrees C. Although the molecular weight of the Fusarium enzyme is significantly lower than the molecular weight of the mammalian enzymes, we have found that pH and temperature optima are similar.

From an economic point of view there exists a clear need for an improved means of producing PAD's in high quantities and in a relatively pure form. A preferred way of doing this is via the overproduction of such a PAD using recombinant DNA techniques. A particularly preferred way of doing this is via the overproduction of an Fusarium derived PAD and a most preferred way of doing this is via the overproduction of an Fusarium graminearum derived PAD. To enable the latter production route unique sequence information of an Fusarium derived peptidyl arginine deiminase is essential. More preferable the whole nucleotide sequence of the encoding gene has to be available.

An improved means of producing the newly identified secreted PAD in high quantities and a relatively pure form is via the overproduction of the Fusarium encoded enzyme using recombinant DNA techniques. A preferred way of doing this is via the overproduction of such a secreted PAD in a food grade host microorganism. Well known food grade microrganisms include Aspergilli, Trichoderma, Streptomyces, Bacilli and yeasts such as Saccharomyces and Kluyveromyces. An even more preferred way of doing this is via overproduction of the secreted Fusarium derived PAD in a food grade fungus such as Aspergillus. Most preferred is the over production of the secreted PAD in a food grade fungus in which the codon use of the PAD-encoding gene has been optimized for the food grade expression host used. In general, to enable the latter optimization routes, unique sequence information of a secreted PAD is desirable. More preferable the whole nucleotide sequence of the PAD encoding gene has to be available. Once the new enzyme has been made available in large quantities and in a relatively pure form, citrulline containing food proteins or hydrolysates of such citrulline containing food proteins are producible in a food grade and economic way. Preferably such citrulline containing food proteins are obtained from food proteins or hydrolysates of such food proteins containing a high percentage of protein-bound arginine. Preferred substrate proteins for the PAD according to the invention have a arginine to citrulline ratio of at least 10:1 (mol/mol), preferably such substrate proteins have an arginine to citrulline ratio of at least of 100:1 (mol/mol) and most preferably the substrate protein will contain no citrulline at all. Preferred substrate proteins for the PAD according to the invention contain at least 3 mol % of protein-bound arginine, more preferably they contain at least 6 mol % of protein-bound arginine. Examples of such substrate proteins are commercially available food proteins from animal origin, such as (skim) milk protein, whey protein, casein or egg protein. Other examples of such substrate proteins are commercially available food proteins from vegetable origin such as cereal protein, potato protein, soy protein, pea protein, rice protein, pea protein as well as proteins from other vegetable sources known to be rich in arginine such as lupins, sesame, palm pits etc. Examples of cereal protein are wheat or maize or fractions thereof for example wheat gluten. Examples of microbial protein is yeast extract or single cell protein for meat replacers. To compensate for the relative shortage of specific amino acids in these arginine-rich proteins or peptides, the nutritional composition of the citrulline containing protein, peptides or hydrolysates can be optimized by adding selected free amino acids or proteins, peptides or hydrolysates that are relatively rich in the amino acids that are under represented in the citrullinated material. Preferred amino acids for enhancing the nutritional value of the citrulline containing protein, peptides or hydrolysates are cysteine, histidine, isoleucine, glutamine and lysine. Alternatively, the citrulline containing protein, peptides or hydrolysates can be mixed with protein, peptides or hydrolysates obtained from protein sources which are relatively rich in these amino acids such as casein, potato, wheat or soy protein. Especially the process of the present invention is useful to modify enzymes resulting in enzymes having modified characteristics such as activity or stability.

In case mammalian tissue is used this tissue is preferably non-human tissue. In case mammalian protein is used, this protein is preferably not blood protein, nerve tissue, brains, organs, muscles or hairs. Preferably the substrate protein used according to the present invention is vegetable protein, skim (milk) protein, whey protein, casein protein, gelatin protein, egg protein or microbial protein.

Another application would be the creation of hydrolysates of such citrulline containing food proteins. These hydrolysates can be produced according to methods that are known in the art. Alternatively the proteins of animal or vegetable origin can be hydrolysed first to obtain a protein hydrolysate and subsequently this hydrolysate can be incubated with the PAD according to the invention.

To provide peptide-bound citrulline in a more concentrated form, arginine enriched hydrolysates can be used as a substrate for the PAD enzyme. In this approach an arginine rich protein source such as, for example, rice protein or pea protein, is first hydrolysed by a suitable endoprotease such as a subtilisin (EC3.4.21.62) or a mucorpepsin (EC3.4.23.23) or a proline-specific protease (EC3.4.21.26) and the resulting hydrolysate is then enriched for arginine containing peptides by using chromatography. In such a chromatographic separation technique, use is made of the positive charge of the arginine residues under certain pH conditions. A practical background on the use of these characteristics in peptide purification can be found in a.o. the Protein Purification Handbook (issued by Amersham Pharmacia Biotech, nowadays GE Healthcare Bio-Sciences, Diegem, Belgium). The resulting arginine enriched peptides are then incubated with the PAD enzyme to obtain the citrulline comprising hydrolysates according to the invention. In another approach towards obtaining arginine enriched hydrolysates, the arginine rich protein source is first incubated with the arginine-specific endoprotease trypsin (EC3.4.21,4). After hydrolysis, the pH of the incubation is adjusted to a value where the water solubility of the protein substrate is minimal so that the larger, non-hydrolysed peptides will precipitate and the smaller, arginine-rich peptides will remain in solution. For example, at near neutral pH, the solubility of pea protein is quite low. By incubating a suspension of pea protein at neutral pH with trypsin (for maximal activity trypsin requires a near neutral pH) only those parts of the pea protein rich in arginine residues will go into solution and a subsequent decantation or filtration step will yield the arginine rich peptides in the supernatant or filtrate respectively. Adding the PAD according to the invention to this supernatant or filtrate will yield the desired high concentration of citrulline containing peptides.

The present invention relates to novel nutraceutical compositions comprising the present protein or protein hydrolysates. The nutraceutical composition comprises protein hydrolysates as the active ingredients for, for example, prevention of high blood pressure and for recovering from malnutrition or from intestinal diseases which comprises administering to a subject in need of such treatment protein, peptide or protein hydrolysate of the present invention.

The present protein, protein hydrolysate, peptide or mixture of peptides comprising citrulline can be used in any suitable form such as solid products, semi solid products (paste) or liquid products such as beverages. For example, a product comprising elevated levels of citrulline is used as a dietary supplement or as a food, beverage, feed or pet food ingredient. A product comprising elevated levels of citrulline also can be used in the form of a personal care application including topical applications in the form of a lotion, gel or an emulsion. In all these applications, the proteins, peptides or hydrolysates may be co-formulated with multi-vitamin preparations comprising vitamins such as vitamin A or vitamin C, with trace elements such as zinc and with minerals which are essential for the maintenance of normal metabolic function but are not synthesized in the body. Furthermore, the citrullinated proteins, peptides or hydrolysates may be combined with specific fatty acids, specific amino acids such as glutamine or proteins or protein hydrolysates enriched in specific amino acids. Also combinations with polyphenols such resveratrol or EGCG, glycosylated and deglycosylated soy isoflavones, prebiotics or probiotics are foreseen.

The term nutraceutical as used herein denotes the usefulness in both the nutritional and pharmaceutical field of application. Thus, the novel nutraceutical compositions can find use as supplement to food and beverages, and as pharmaceutical formulations or medicaments for enteral or parenteral application which may be solid formulations such as capsules or tablets, or liquid formulations, such as solutions or suspensions. As will be evident from the foregoing, the term nutraceutical composition also comprises food and beverages comprising the present peptide containing composition and optionally carbohydrate as well as supplement compositions, for example dietary supplements, comprising the aforesaid citrullinated protein, peptides or hydrolysates.

The term dietary supplement as used herein denotes a product taken by mouth that contains a “dietary ingredient” intended to supplement the diet. The “dietary ingredients” in these products may include: vitamins, minerals, herbs or other botanicals, amino acids, and substances such as enzymes, organ tissues, glandules, and metabolites. Dietary supplements can also be extracts or concentrates, and may be found in many forms such as tablets, capsules, softgels, gelcaps, liquids, or powders. They can also be in other forms, such as a bar, but if they are, information on the label of the dietary supplement will in general not represent the product as a conventional food or a sole item of a meal or diet. By hydrolysate, protein hydrolysate or hydrolysed protein is meant the product that is formed by a proteolytic hydrolysis of the substrate protein. Preferably the hydrolysis is an enzymatic hydrolysis. A soluble hydrolysate being the (water) soluble fraction of the protein hydrolysate which is also described herein as soluble peptide containing composition or composition comprising soluble peptides, or a mixture of a protein hydrolysate and a soluble hydrolysate.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” or “protein” is defined herein as a chain comprising of more than 30 amino acid residues. All (oligo)peptide and polypeptide formulas or sequences herein are written from left to right in the direction from amino-terminus to carboxy-terminus, in accordance with common practice. The one-letter code of amino acids used herein is commonly known in the art and can be found in Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989).

There are twenty “standard” amino acids used by cells in protein biosynthesis which are specified by the general genetic code. In the present application by amino acids is meant these twenty standard amino acids and citrulline.

Preferably the isolated dipeptide citrulline-arginine is riot part of the invention. However, hydrolysates comprising this dipeptide and the use of such hydrolysates are a further embodiment of the present invention. Use of the isolated dipeptide citrulline-arginine as active ingredient for recovering from malnutrition or from intestinal diseases also is an object of the present invention. Products in which Irish moss or extracts or other products obtained from Irish moss is used, are not part of the present invention.

Proteins or peptides incorporating large amounts of citrulline can offer significant benefits. Most importantly they offer for the first time the possibility to make peptide bound citrulline available for non-medical applications such as special food, infant nutrition or nutritional supplements for special consumer groups. Additionally such proteins or peptides offer an organoleptical benefit because proteins and protein hydrolysates comprising citrulline residues do not exhibit bitter taste. Moreover the citrulline according to the invention is present as peptide bound citrulline in contrast to free citrulline, which may not be acceptable according to some national legislation. As a result, the process according to the invention allows for the first time the production of citrulline containing foods, supplements and clinical products with an acceptable taste profile.

Another important advantage of the proteins or peptides according to the invention is that they might exhibit a reduced allergenicity. All major food proteins, such as milk and its casein and whey protein fractions as well as vegetable protein fractions obtained from, for example, soy isolates, rice proteins and wheat gluten are considered important antigenic compounds. Usually protein antigenicity is overcome by hydrolyzing the proteins to peptides having less than 8-10 amino acid residues. However, the hydrolysates created by such an extensive proteolytic digestion exhibit disadvantages that may include bitterness, brothy off-flavours and increased osmotic values. The economic importance of protein antigenicity is illustrated by the fact that the prevalence of food allergies and asthma in infants and young children is growing. For example, cow's milk allergy affects 2.5% of children under 3 years of age. Cow milk allergy is often encountered during the first months of life and within a week after the introduction of cow milk. Anticipating to cow milk allergy are various infant formula products incorporating cow milk protein or cow milk fractions hydrolysed to different degrees. We have found that by subjecting food proteins to an incubation with the PAD according to the invention, the antigenicity or allergenicity of the resulting citrullinated protein or protein hydrolysate is reduced. Most importantly this effect is obtained without the negative effects connected with extensive proteolysis such as an impaired taste or reducing emulsifying capacities. In view of the rising number of sports people, people of high age and people suffering from a food allergy, the products according to the invention will be of great economic importance. For all these target groups, the improved taste is an aspect of significant psychological importance. Hitherto the production of such foods or supplements on an industrial scale and in an economic way was not possible. Because the currently industrially available food proteins and/or food protein hydrolysates do not incorporate protein- or peptide bound citrulline residues, proteins or protein hydrolysates or peptides enriched in citrulline residues are new. The term “enriched” is intended to mean that at least 15%, preferably at least 30%, more preferably at least 45%, still more preferably at least 60% and most preferably at least 80% of the arginine residues which were originally present in the protein or protein hydrolysate is transformed into a citrulline residue. Therefore, in the product of the present process i.e. the citrullinated protein, the hydrolysate of the citrullinated protein or the citrullinated hydrolysate, at least 15%, preferably at least 30%, more preferably at least 45%, still more preferably at least 60% and most preferably at least 80% of the arginine residues present have been converted into citrulline residues. The amino acid analysis method used for establishing the amount of arginine or citrulline residues present is specified in the Materials & Methods section of this application. Important to note is that as an artefact of the acid hydrolysis process typically used to liberate free amino acids during amino acid analysis, part of the newly generated citrulline residues is converted into ornithine residues. To calculate the level of citrulline residues present, the levels of citrulline and the ornithine residues present have to be added up.

To obtain citrulline containing protein hydrolysates, the citrulline containing protein can be enzymatically hydrolysed. Alternatively an existing protein hydrolysate, i.e. an hydrolysate not comprising peptide-bound citrulline, can be incubated with the PAD according to the invention to obtain a protein hydrolysate comprising peptide-bound citrulline. Protein hydrolysates can be produced using hydrolysis methods known in the art. Preferably such hydrolysates have Degrees of Hydrolysis (DR) between 5 and 50, more preferably between 10 and 35. The method for establishing DH values is specified in the Materials & Methods section of this application. The protein hydrolysates comprising citrulline according to the invention can be used in infant and clinical nutrition, in therapeutic diets as well as in consumer diets and sport nutrition. Also new are food or diet or clinical products incorporating such citrullinated proteins, protein hydrolysates or peptides. Furthermore the proteins or protein hydrolysates comprising protein- or peptide-bound citrulline can be used in various topical applications including personal care applications and in nutritional products for animals and pets.

The proteins, peptides or protein hydrolysates comprising citrulline according to the invention can be used in many new and surprising applications. Basically, the proteins, peptides or protein hydrolyzates comprising citrulline can be used in all applications in which suppletion of free arginine has been shown to be beneficial. Therefore, in all conditions characterized by an arginine-deficient state, it is expected that proteins, peptides or protein hydrolysates comprising citrulline according to the invention, will reduce this arginine-deficient state. Generally, proteins, peptides or protein hydrolysates comprising citrulline offer advantages to sustain protein metabolism in individuals recovering from malnutrition or from intestinal diseases, such as short bowel syndrome or from protein-energy malnutrition as the result of ageing. Furthermore proteins, peptides or protein hydrolysates comprising citrulline can play an important role in maintaining the health of the gastrointestinal tract. Because arginine as well as citrulline are precursors for nitric oxide production and thus vasodilatation, the adequate supply of these amino acids plays an important role in preventing the risks of a variety of vascular diseases. In the scientific literature this has been demonstrated for amongst others peripheral arterial disease, graft coronary artery disease and asthma. Thus, proteins, peptides or protein hydrolysates comprising citrulline are of special interest as additions to diets preventing these diseases as well as in the prevention of pressure ulcers or in improving pressure ulcer healing. Moreover, the proteins, peptides and protein hydrolysates according to the invention also are particularly useful in therapeutic regimens for a.o. artherosclerosis, angina pectoris, hypertension, coronary heart disease, Type II diabetes mellitus to decrease insulin and glucose concentrations and in the treatment of HIV infections, burns, trauma and cancer. The use of proteins, peptides or protein hydrolysates comprising citrulline for the treatment of sarcopenia is particularly noteworthy. Furthermore, the use of proteins, peptides or protein hydrolysates comprising citrulline in pre-operative diets to improve the immunological status, especially under stress conditions, or to ameliorate micro-circulatory hypo-perfusion of patients. Because sepsis is a major health problem with a high mortality rate, the beneficial effects of diets incorporating proteins, peptides or protein hydrolysates comprising citrulline in the prevention of sepsis also is important to mention. Because of their hypoammonaemic effect, the proteins, peptides or protein hydrolysates comprising citrulline can also be used in cases of a disturbed urea cycle, e.g. in patients suffering from inherited enzyme defects or to reduce pressure on glomular function of the kidneys of persons suffering from renal failure. The proteins, peptides and protein hydrolysates according to the invention also can be used in products destined for consumers with non-medical needs, for example athletes or sport people. Especially, because citrulline is expected to improve blood flow by stimulating eNOS NO production, it is expected that exercise performance could be improved via improvement of muscle blood flow. More recently, evidence is being collected that indicates that an inhibition of NO synthesis causes hyperlipidemia and fat accretion. Therefore dietary supplementation with peptide bound citrulline according to the invention may aid in the prevention and treatment of metabolic syndrome in obese humans and animals, such as pets. Products in this category include fortified fruit juices and sports drinks to fight the feeling of fatigue and to enhance physical endurance and recovery after prolonged high intensity exercise. Another important application is the use of the proteins, peptides and protein hydrolysates according to the invention in infant formula, e.g. for infants that are allergic, for babies developing allergic reactions or for non-allergic infants where the citrullinated proteins or peptides act to delay or prevent cow milk sensitization. In topical applications the citrullinated proteins or peptides are surprisingly effective in scavenging of hydroxyl radicals and to control and counter act active oxygen species. Moreover, the active PAD according to the invention can be used for direct topical application to improve the epidermal keratinization or to fight signs of psoriasis. A useful way of applying the active enzyme to the skin is described in U.S. Pat. No. 6,117,433. Another new application of the active PAD according to the invention is the incorporation in all kinds of doughs as it has been observed that this increases the taste and the odor of the baked goods obtained. The active PAD according to the invention can also be used to reduce the levels of the carcinogen ethyl carbamate in fermented foods and beverages such as wine, beer and spirits by reducing the amount of urea in such fermented foods and beverages.

A polypeptide of the invention which has peptidyl arginine deiminase activity may be in an isolated form. As defined herein, an isolated polypeptide is an endogenously produced or a recombinant polypeptide which is essentially free from other non-peptidyl arginine deiminase polypeptides, and is typically at least about 20% pure, preferably at least about 40% pure, more preferably at least about 60% pure, even more preferably at least about 80% pure, still more preferably about 90% pure, and most preferably about 95% pure, as determined by SDS-PAGE. The polypeptide may be isolated by centrifugation and chromatographic methods, or any other technique known in the art for obtaining pure proteins from crude solutions. It will be understood that the polypeptide may be mixed with carriers or diluents which do not interfere with the intended purpose of the polypeptide, and thus the polypeptide in this form will still be regarded as isolated. It will generally comprise the polypeptide in a preparation in which more than 20%, for example more than 30%, 40%, 50%, 80%, 90%, 95% or 99%, by weight of the proteins in the preparation is a polypeptide of the invention.

Preferably, the polypeptide of the invention is obtainable from a microorganism which possesses a gene encoding an enzyme with peptidyl arginine deiminase activity. More preferably polypeptide of the invention is secreted from this microorganism. Even more preferably the microorganism is fungal, and optimally is a filamentous fungus. Preferred organisms are thus of the genus Fusarium, such as those of the species Fusarium graminearum.

In a first embodiment, the present invention provides an isolated polypeptide having an amino acid sequence which has a degree of amino acid sequence identity to amino acids 1to 640 of SEQ ID NO: 6 (i.e. the polypeptide) of at least 30%, preferably at least 40%, preferably at least 50%, preferably at least 60%, preferably at least 70%, more preferably at least 80%, even more preferably at least 90%, still more preferably at least 95%, and most preferably at least 97%, and which has peptidyl arginine deiminase activity.

For the purposes of the present invention, the degree of identity between two or more amino acid sequences is determined by BLAST P protein database search program (Altschul et al., 1997, Nucleic Acids Research 25. 3389-3402) with matrix Blosum 62 and an expected threshold of 10.

A polypeptide of the invention may comprise the amino acid sequence set forth in SEQ ID NO: 6 or a substantially homologous sequence, or a fragment of either sequence having peptidyl arginine deiminase activity. In general, the naturally occurring amino acid sequence shown in SEQ ID NO: 6 is preferred.

The polypeptide of the invention may also comprise a naturally occurring variant or species homologue of the polypeptide of SEQ ID NO: 6.

A variant is a polypeptide that occurs naturally in, for example, fungal, bacterial, yeast or plant cells, the variant having peptidyl arginine deiminase activity and a sequence substantially similar to the protein of SEQ ID NO: 6. The term “variants” refers to polypeptides which have the same essential character or basic biological functionality as the peptidyl arginine deiminase of SEQ ID NO: 6, and includes allelic variants. Preferably, a variant polypeptide has at least the same level of peptidyl arginine deiminase activity as the polypeptide of SEQ ID NO: 6. Variants include allelic variants either from the same strain as the polypeptide of SEQ ID NO: 6. or from a different strain of the same genus or species. Examples of variants of the polypeptide of SEQ ID NO: 6 are listed in SEQ ID NO: 7.

Similarly, a species homologue of the inventive protein is an equivalent protein of similar sequence which is an peptidyl arginine deiminase and occurs naturally in another species. Examples of species homologues of the polypeptide of SEQ ID NO: 6 are listed in SEQ ID NO: 8-10, 13 and 14.

Variants and species homologues can be isolated using the procedures described herein which were used to isolate the polypeptide of SEQ ID NO: 6 and performing such procedures on a suitable cell source, for example a bacterial, yeast, fungal or plant cell. Also possible is to use a probe of the invention to probe libraries made from yeast, bacterial, fungal or plant cells in order to obtain clones expressing variants or species homologues of the polypepetide of SEQ ID NO: 6. The methods that can be used to isolate variants and species homologues of a known gene are extensively described in literature, and known to those skilled in the art. These genes can be manipulated by conventional techniques to generate a polypeptide of the invention which thereafter may be produced by recombinant or synthetic techniques known per se.

The sequence of the polypeptide of SEQ ID NO: 6 and of variants and species homologues can also be modified to provide polypeptides of the invention. Amino acid substitutions may be made, for example from 1, 2 or 3 to 10, 20 or 30 substitutions. The same number of deletions and insertions may also be made. These changes may be made outside regions critical to the function of the polypeptide, as such a modified polypeptide will retain its peptidyl arginine deiminase activity.

Polypeptides of the invention include fragments of the above mentioned full length polypeptides and of variants thereof, including fragments of the sequence set out in SEQ ID NO: 6. Such fragments will typically retain activity as an peptidyl arginine deiminase. Fragments may be at least 50, 100 or 200 amino acids long or may be this number of amino acids short of the full length sequence shown in SEQ ID NO: 6.

Polypeptides of the invention can, if necessary, be produced by synthetic means although usually they will be made recombinantly as described below Synthetic polypeptides may be modified, for example, by the addition of histidine residues or a T7 tag to assist their identification or purification, or by the addition of a signal sequence to promote their secretion from a cell.

Thus, the variants sequences may comprise those derived from strains of Fusarium other than the strain from which the polypeptide of SEQ ID NO: 6 was isolated. Variants can be identified from other Fusarium strains by looking for peptidyl arginine deiminase activity and cloning and sequencing as described herein. Variants may include the deletion, modification or addition of single amino acids or groups of amino acids within the protein sequence, as long as the peptide maintains the basic biological functionality of the peptidyl arginine deiminase of SEQ ID NO: 6.

Amino acid substitutions may be made, for example from 1, 2 or from 3 to 10, 20 or 30 substitutions. The modified polypeptide will generally retain activity as a peptidyl arginine deiminase. Conservative substitutions may be made; such substitutions are well known in the art.

Shorter polypeptide sequences are within the scope of the invention. For example, a peptide of at least 50 amino acids or up to 60, 70, 80, 100, 150 or 200 amino acids in length is considered to fall within the scope of the invention as long as it demonstrates the basic biological functionality of the peptidyl arginine deiminase of SEQ ID NO: 6. In particular, but not exclusively, this aspect of the invention encompasses the situation in which the protein is a fragment of the complete protein sequence.
The present invention also relates to a polynucleotide which encodes a polypeptide which has protein arginine deiminase activity said polynucleotide comprises
(a) a polynucleotide sequence which encodes amino acid SEQ ID NO: 11, and
(b) a polynucleotide sequence which encodes a pre-protein signal sequence whereby the encoded pre-protein signal sequence is located at the amino terminus of the encoded pre-polypeptide and is preferably 15 to 30 amino acids in length.

For the present invention polypeptides that contain the PAD consensus sequence of SEQ ID NO: 11 are within the invention. Preferably polypeptides that contain both the PAD consensus sequence of SEQ ID NO: 11 and are encoded as a pre-protein containing a signal sequence, are part of the invention. For the present invention it is especially relevant that the protein of interest is actively secreted into the growth medium. Secreted proteins are normally originally synthesized as pre-proteins and the pre-sequence (signal sequence) is subsequently removed during the secretion process. The secretion process is basically similar in prokaryotes and eukaryotes: the actively secreted pre-protein is threaded through a membrane, the signal sequence is removed by a specific signal peptidase, and the mature protein is (re)-folded. Also for the signal sequence a general structure can be recognized. Signal sequences for secretion are located at the amino-terminus of the pre-protein, and are generally 15-30 amino-acids in length. The amino-terminus preferably contains positively charged amino-acids, and preferably no acidic amino-acids. It is thought that this positively charged region interacts with the negatively charged head groups of the phospholipids of the membrane. This region is followed by a hydrophobic, membrane-spanning core region. This region is generally 10-20 amino-acids in length and consists mainly of hydrophobic amino-acids. Charged amino-acids are normally not present in this region. The membrane spanning region is followed by the recognition site for signal peptidase. The recognition site consists of amino-acids with the preference for small-X-small. Small amino-acids can be alanine, glycine, serine or cysteine. X can be any amino acids. Using such rules an algorithm has been written that is able to recognize such signal sequences from eukaryotes and prokaryotes (Bendtsen, Nielsen, von Heijne and Brunak. (2004) J. Mol. Biol., 340:783-795). The SignalP program to calculate and recognize signal sequences in proteins is generally available (http://www.cbs.dtu.dk/services/SignalP/).

Relevant for the present invention is that signal sequences can be recognized from the deduced protein sequence of a sequenced gene. If a gene encodes a protein where a signal sequence is predicted using the SignalP program, the chance that this protein is secreted is high. The purpose of this invention is therefore to provide a new method to find new proteins that have PAD activity using the consensus of SEQ ID NO: 11, in combination with the presence of a signal sequence detected by the SignalP program.

In a second embodiment, the present invention provides an isolated polypeptide which has peptidyl arginine deiminase activity, and is encoded by polynucleotides which hybridize or are capable of hybrizing under low stringency conditions, more preferably medium stringency conditions, and most preferably high stringency conditions, with (I) the nucleic acid sequence of SEQ ID NO: 3 or a nucleic acid fragment comprising at least the c-terminal portion of SEQ ID NO: 3, but having less than all or having bases differing from the bases of SEQ ID NO: 3; or (ii) with a nucleic acid strand complementary to SEQ ID NO: 3.

The term “capable of hybridizing” means that the target polynucleotide of the invention can hybridize to the nucleic acid used as a probe (for example, the nucleotide sequence set forth in SEQ ID NO: 3, or a fragment thereof, or the complement of SEQ ID NO: 3, or a fragment thereof) at a level significantly above background. The invention also includes the polynucleotides that encode the peptidyl arginine deiminase of the invention, as well as nucleotide sequences which are complementary thereto. The nucleotide sequence may be RNA or DNA, including genomic DNA, synthetic DNA or cDNA. Preferably, the nucleotide sequence is DNA and most preferably, a genomic DNA sequence. Typically, a polynucleotide of the invention comprises a contiguous sequence of nucleotides which is capable of hybridizing under selective conditions to the coding sequence or the complement of the coding sequence of SEQ ID NO: 3. Such nucleotides can be synthesized according to methods well known in the art.

A polynucleotide of the invention can hybridize to the coding sequence or the complement of the coding sequence of SEQ ID NO: 3 at a level significantly above background. Background hybridization may occur, for example, because of other cDNAs present in a cDNA library. The signal level generated by the interaction between a polynucleotide of the invention and the coding sequence or complement of the coding sequence of SEQ ID NO; 3 is typically at least 10 fold, preferably at least 20 fold, more preferably at least 50 fold, and even more preferably at least 100 fold, as intense as interactions between other polynucleotides and the coding sequence of SEQ ID NO: 3. The intensity of interaction may be measured, for example, by radiolabelling the probe, for example with ³²P. Selective hybridization may typically be achieved using conditions of low stringency (0.3M sodium chloride and 0.03M sodium citrate at about 40° C.), medium stringency (for example, 03.M sodium chloride and 0.03M sodium citrate at about 50° C.) or high stringency (for example, 0.3M sodium chloride and 0.03M sodium citrate at about 60° C.).

A polynucleotide of the invention also includes synthetic genes that can encode for the polypeptide of SEQ ID NO: 6 or variants thereof. It is sometimes preferable to adapt the codon usage of a gene to the preferred bias in a production host. Techniques to design and construct synthetic genes are generally available (i.e http://www.dnatwopointo.com/).

Modifications

Polynucleotides of the invention may comprise DNA or RNA. They may be single or double stranded. They may also be polynucleotides which include within them synthetic or modified nucleotides including peptide nucleic acids. A number of different types of modifications to polynucleotides are known in the art. These include a methylphosphonate and phosphorothioate backbones, and addition of acridine or polylysine chains at the 3′ and/or 5′ ends of the molecule. For the purposes of the present invention, it is to be understood that the polynucleotides described herein may be modified by any method available in the art.

It is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The coding sequence of SEQ ID NO: 3 may be modified by nucleotide substitutions, for example from 1, 2 or 3 to 10, 25, 50, 100, or more substitutions. The polynucleotide of SEQ ID NO: 3 may alternatively or additionally be modified by one or more insertions and/or deletions and/or by an extension at either or both ends. The modified polynucleotide generally encodes a polypeptide which has peptidyl arginine deiminase activity. Degenerate substitutions may be made and/or substitutions may be made which would result in a conservative amino acid substitution when the modified sequence is translated, for example as discussed with reference to polypeptides later.

Homologues

A nucleotide sequence which is capable of selectively hybridizing to the complement of the DNA coding sequence of SEQ ID NO: 3 is included in the invention and will generally have at least 50% or 60%, at least 70%, at least 80%, at least 90%, at least 95%, at least 98% or at least 99% sequence identity to the coding sequence of SEQ ID NO: 3 over a region of at least 60, preferably at least 100, more preferably at least 200 contiguous nucleotides or most preferably over the full length of SEQ ID NO: 3. Likewise, a nucleotide which encodes an active peptidyl arginine deiminase and which is capable of selectively hybridizing to a fragment of a complement of the DNA coding sequence of SEQ ID NO: 3, is also embraced by the invention. A C-terminal fragment of the nucleic acid sequence of SEQ ID NO: 3 which is at least 80% or 90% identical over 60, preferably over 100 nucleotides, more preferably at least 90% identical over 200 nucleotides is encompassed by the invention.

Any combination of the above mentioned degrees of identity and minimum sizes may be used to define polynucleotides of the invention, with the more stringent combinations (i.e. higher identity over longer lengths) being preferred. Thus, for example, a polynucleotide which is at least 80% or 90% identical over 60, preferably over 100 nucleotides, forms one aspect of the invention, as does a polynucleotide which is at least 90% identical over 200 nucleotides.

The UWGCG Package provides the BESTFIT program which may be used to calculate identity (for example used on its default settings).

The PILEUP and BLAST N algorithms can also be used to calculate sequence identity or to line up sequences (such as identifying equivalent or corresponding sequences, for example on their default settings).

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold. These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix alignments (B) of 50, expectation (E) of 10, M=5, N=4, and a comparison of both strands.

The BLAST algorithm performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001

Primers and Probes

Polynucleotides of the invention include and may be used as primers, for example as polymerase chain reaction (PCR) primers, as primers for alternative amplification reactions, or as probes for example labelled with a revealing label by conventional means using radioactive or non-radioactive labels, or the polynucleotides may be cloned into vectors. Such primers, probes and other fragments will be at least 15, for example at least 20, 25, 30 or 40 nucleotides in length. They will typically be up to 40, 50, 60, 70, 100, 150, 200 or 300 nucleotides in length, or even up to a few nucleotides (such as 5 or 10 nucleotides) short of the coding sequence of SEQ ID NO: 5.

In general, primers will be produced by synthetic means, involving a step-wise manufacture of the desired nucleic acid sequence one nucleotide at a time. Techniques for accomplishing this and protocols are readily available in the art. Longer polynucleotides will generally be produced using recombinant means, for example using PCR cloning techniques. This will involve making a pair of primers (typically of about 15-30 nucleotides) to amplify the desired region of the peptidyl arginine deiminase to be cloned, bringing the primers into contact with mRNA, cDNA or genomic DNA obtained from a yeast, bacterial, plant, prokaryotic or fungal cell, preferably of an Fusarium strain, performing a polymerase chain reaction under conditions suitable for the amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. Such PCR cloning of variants of the PAD gene of SEQ ID NO: 3 was performed within this invention and the DNA sequences of these genes are mentioned in SEQ ID NO: 4, and the deduced protein sequences are mentioned in SEQ ID NO: 7.

Alternatively, synthetic genes can be constructed that encompass the coding region of the secreted peptidyl arginine deiminase or variants thereof. Polynucleotides that are altered in many positions, but still encode the same protein can be conveniently be designed and constructed using these techniques. This has as advantage that the codon usage can be adapted to the preferred expression host, so productivity of the protein in this host can be improved. Also the polynucleotide sequence of a gene can be changed to improve mRNA stability or reduced turnover. This can lead to improved expression of the desired protein or variants thereof. Additionally, the polynucleotide sequence can be changed in a synthetic gene such that mutations are made in the protein sequence that have a positive effect on secretion efficiency, stability, proteolytic vulnerability, temperature optimum, specific activity or other relevant properties for industrial production or application of the protein. Companies that provide services to construct synthetic genes and optimize codon usage are generally available.

Such techniques may be used to obtain all or part of the polynucleotides encoding the peptidyl arginine deiminase sequences described herein. Introns, promoter and trailer regions are within the scope of the invention and may also be obtained in an analogous manner (e.g. by recombinant means, PCR or cloning techniques), starting with genomic DNA from a fungal, yeast, bacterial plant or prokaryotic cell.

The polynucleotides or primers may carry a revealing label. Suitable labels include radioisotopes such as ³²P or ³⁵S, fluorescent labels, enzyme labels, or other protein labels such as biotin. Such labels may be added to polynucleotides or primers of the invention and may be detected using techniques known to persons skilled in the art.

Polynucleotides or primers (or fragments thereof) labelled or unlabelled may be used in nucleic acid-based tests for detecting or sequencing a peptidyl arginine deiminase or a variant thereof in a fungal sample. Such detection tests will generally comprise bringing a fungal sample suspected of containing the DNA of interest into contact with a probe comprising a polynucleotide or primer of the invention under hybridizing conditions, and detecting any duplex formed between the probe and nucleic acid in the sample, Detection may be achieved using techniques such as PCR or by immobilizing the probe on a solid support, removing any nucleic acid in the sample which is not hybridized to the probe, and then detecting any nucleic acid which is hybridized to the probe. Alternatively, the sample nucleic acid may be immobilized on a solid support, the probe hybridized and the amount of probe bound to such a support after the removal of any unbound probe detected.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridizing the probe to nucleic acid in the sample, control reagents, instructions, and the like. The probes and polynucleotides of the invention may also be used in microassay.

Preferably, the polynucleotide of the invention is obtainable from the same organism as the polypeptide, such as a fungus, in particular a fungus of the genus Fusarium.

Production of Polynucleotides

Polynucleotides which do not have 100% identity with SEQ ID NO: 3 but fall within the scope of the invention can be obtained in a number of ways. Thus, variants of the peptidyl arginine deiminase sequence described herein may be obtained for example, by probing genomic DNA libraries made from a range of organisms, such as those discussed as sources of the polypeptides of the invention. In addition, other fungal, plant or prokaryotic homologues of peptidyl arginine deiminase may be obtained and such homologues and fragments thereof in general will be capable of hybridising to SEQ ID NO: 3. Such sequences may be obtained by probing cDNA libraries or genomic DNA libraries from other species, and probing such libraries with probes comprising all or part of SEQ ID NO: 3 under conditions of low, medium to high stringency (as described earlier). Nucleic acid probes comprising all or part of SEQ ID NO: 3 may be used to probe cDNA or genomic libraries from other species, such as those described as sources for the polypeptides of the invention.

Species homologues may also be obtained using degenerate PCR, which uses primers designed to target sequences within the variants and homologues which encode conserved amino acid sequences. The primers can contain one or more degenerate positions and will be used at stringency conditions lower than those used for cloning sequences with single sequence primers against known sequences. A preferable way to obtain species homologues of PAD is to design primers that target sequences that encode the consensus sequence described in SEQ ID NO: 11.

Alternatively, such polynucleotides may be obtained by site directed mutagenesis of the peptidyl arginine deiminase sequences or variants thereof. This may be useful where, for example, silent codon changes to sequences are required to optimize codon preferences for a particular host cell in which the polynucleotide sequences are being expressed. Other sequence changes may be made in order to introduce restriction enzyme recognition sites, or to alter the property or function of the polypeptides encoded by the polynucleotides.

The invention includes double stranded polynucleotides comprising a polynucleotide of the invention and its complement.

The present invention also provides polynucleotides encoding the polypeptides of the invention described above. Since such polynucleotides will be useful as sequences for recombinant production of polypeptides of the invention, it is not necessary for them to be capable of hybridising to the sequence of SEQ ID NO: 3, although this will generally be desirable. Otherwise, such polynucleotides may be labelled, used, and made as described above if desired.

Recombinant Polynucleotides.

The invention also provides vectors comprising a polynucleotide of the invention, including cloning and expression vectors, and in another aspect methods of growing, transforming or transfecting such vectors into a suitable host cell, for example under conditions in which expression of a polypeptide oft or encoded by a sequence of, the invention occurs. Provided also are host cells comprising a polynucleotide or vector of the invention wherein the polynucleotide is heterologous to the genome of the host cell. The term “heterologous”, usually with respect to the host cell, means that the polynucleotide does not naturally occur in the genome of the host cell or that the polypeptide is not naturally produced by that cell. Preferably, the host cell is a yeast cell, for example a yeast cell of the genus Kluyveromyces, Pichia, Hansenula or Saccharomyces or a filamentous fungal cell, for example of the genus Aspergillus, Trichoderma or Fusarium.

Vectors

The vector into which the expression cassette of the invention is inserted may be any vector that may conveniently be subjected to recombinant DNA procedures, and the choice of the vector will often depend on the host cell into which it is to be introduced. Thus, the vector may be an autonomously replicating vector, i.e. a vector which exists as an extra-chromosomal entity, the replication of which is independent of chromosomal replication, such as a plasmid. Alternatively, the vector may be one which, when introduced into a host cell, is integrated into the host cell genome and replicates together with the chromosome(s) into which it has been integrated.

Preferably, when a polynucleotide of the invention is in a vector it is operably linked to a regulatory sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence such as a promoter, enhancer or other expression regulation signal “operably linked” to a coding sequence is positioned in such a way that expression of the coding sequence is achieved under production conditions.

The vectors may, for example in the case of plasmid, cosmid, virus or phage vectors, be provided with an origin of replication, optionally a promoter for the expression of the polynucleotide and optionally an enhancer and/or a regulator of the promoter. A terminator sequence may be present, as may be a polyadenylation sequence. The vectors may contain one or more selectable marker genes, for example an ampicillin resistance gene in the case of a bacterial plasmid or a neomycin resistance gene for a mammalian vector. Vectors may be used in vitro, for example for the production of RNA or can be used to transfect or transform a host cell.

The DNA sequence encoding the polypeptide is preferably introduced into a suitable host as part of an expression construct in which the DNA sequence is operably linked to expression signals which are capable of directing expression of the DNA sequence in the host cells. For transformation of the suitable host with the expression construct transformation procedures are available which are well known to the skilled person. The expression construct can be used for transformation of the host as part of a vector carrying a selectable marker, or the expression construct is co-transformed as a separate molecule together with the vector carrying a selectable marker. The vectors may contain one or more selectable marker genes.

Preferred selectable markers include but are not limited to those that complement a detect in the host cell or confer resistance to a drug. They include for example versatile marker genes that can be used for transformation of most filamentous fungi and yeasts such as acetamidase genes or cDNAs (the amdS, niaD, facA genes or cDNAs from A.nidulans, A.oryzae, or A.niger), or genes providing resistance to antibiotics like G418, hygromycin, bleomycin, kanamycin, phleomycin or benomyl resistance (benA). Alternatively, specific selection markers can be used such as auxotrophic markers which require corresponding mutant host strains: e.g. URA3 (from S.cerevisiae or analogous genes from other yeasts), pyrG or pyrA (from A.nidulans or A.niger), argB (from A.nidulans or A.niger) or trpC. In a preferred embodiment the selection marker is deleted from the transformed host cell after introduction of the expression construct so as to obtain transformed host cells capable of producing the polypeptide which are free of selection marker genes.

Other markers include ATP synthetase subunit 9 (oliC), orotidine-5′phosphate-decarboxylase (pvrA), the bacterial G418 resistance gene (useful in yeast, but not in filamentous fungi), the ampicillin resistance gene (E. coli), the neomycin resistance gene (Bacillus) and the E. coli uidA gene, coding for glucuronidase (GUS). Vectors may be used in vitro, for example for the production of RNA or to transfect or transform a host cell.

For most filamentous fungi and yeast, the expression construct is preferably integrated into the genome of the host cell in order to obtain stable transformants. However, for certain yeasts suitable episomal vector systems are also available into which the expression construct can be incorporated for stable and high level expression. Examples thereof include vectors derived from the 2 μm, CEN and pKD1 plasmids of Saccharomyces and Kluyveromyces, respectively, or vectors containing an AMA sequence (e.g. AMA1 from Aspergillus). When expression constructs are integrated into host cell genomes, the constructs are either integrated at random loci in the genome, or at predetermined target loci using homologous recombination, in which case the target loci preferably comprise a highly expressed gene. A highly expressed gene is a gene whose mRNA can make up at least 0.01% (w/w) of the total cellular mRNA, for example under induced conditions, or alternatively, a gene whose gene product can make up at least 0.2% (w/w) of the total cellular protein, or, in case of a secreted gene product, can be secreted to a level of at least 0.05 g/l.

An expression construct for a given host cell will usually contain the following elements operably linked to each other in consecutive order from the 5′-end to 3′-end relative to the coding strand of the sequence encoding the polypeptide of the first aspect: (1) a promoter sequence capable of directing transcription of the DNA sequence encoding the polypeptide in the given host cell, (2) preferably, a 5′-untranslated region (leader), (3) optionally, a signal sequence capable of directing secretion of the polypeptide from the given host cell into the culture medium, (4) the DNA sequence encoding a mature and preferably active form of the polypeptide, and preferably also (5) a transcription termination region (terminator) capable of terminating transcription downstream of the DNA sequence encoding the polypeptide.

Downstream of the DNA sequence encoding the polypeptide, the expression construct preferably contains a 3′ untranslated region containing one or more transcription termination sites, also referred to as a terminator. The origin of the terminator is less critical. The terminator can for example be native to the DNA sequence encoding the polypeptide. However, preferably a bacterial terminator is used in bacterial host cells, a yeast terminator is used in yeast host cells and a filamentous fungal terminator is used in filamentous fungal host cells. More preferably, the terminator is endogenous to the host cell in which the DNA sequence encoding the polypeptide is expressed.

Enhanced expression of the polynucleotide encoding the polypeptide of the invention may also be achieved by the selection of heterologous regulatory regions, e.g. promoter, signal sequence and terminator regions, which serve to increase expression and, if desired, secretion levels of the protein of interest from the chosen expression host and/or to provide for the inducible control of the expression of the polypeptide of the invention.

Aside from the promoter native to the gene encoding the polypeptide of the invention, other promoters may be used to direct expression of the polypeptide of the invention. The promoter may be selected for its efficiency in directing the expression of the polypeptide of the invention in the desired expression host.

Promoters/enhancers and other expression regulation signals may be selected to be compatible with the host cell for which the expression vector is designed. For example prokaryotic promoters may be used, in particular those suitable for use in E. coli strains. When expression of the polypeptides of the invention is carried out in mammalian cells, mammalian promoters may be used. Tissues-specific promoters, for example hepatocyte cell-specific promoters, may also be used. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, herpes simplex virus promoters or adenovirus promoters.

Suitable yeast promoters include the S. cerevisiae GAL4 and ADH promoters and the S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular endothelial or neuronal cell specific promoters (for example the DDAHI and DDAHII promoters), are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

A variety of promoters can be used that are capable of directing transcription in the host cells of the invention. Preferably the promoter sequence is derived from a highly expressed gene as previously defined. Examples of preferred highly expressed genes from which promoters are preferably derived and/or which are comprised in preferred predetermined target loci for integration of expression constructs, include but are not limited to genes encoding glycolytic enzymes such as triose-phosphate isomerases (TPI), glyceraldehyde-phosphate dehydrogenases (GAPDH), phosphoglycerate kinases (PGK), pyruvate kinases (PYK), alcohol dehydrogenases (ADH), as well as genes encoding amylases, glucoamylases, proteases, xylanases, cellobiohydrolases, β-galactosidases, alcohol (methanol) oxidases, elongation factors and ribosomal proteins. Specific examples of suitable highly expressed genes include e.g. the LAC4 gene from Kluyveromyces sp., the methanol oxidase genes (AOX and MOX) from Hansenula and Pichia, respectively, the glucoamylase (glaA) genes from A.niger and A.awamori, the A.oryzae TAKA-amylase gene, the A.nidulans gpdA gene and the T.reesei cellobiohydrolase genes.

Examples of strong constitutive and/or inducible promoters which are preferred for use in fungal expression hosts are those which are obtainable from the fungal genes for xylanase (xlnA), phytase, ATP-synthetase subunit 9 (oliC), triose phosphate isomerase (tpi), alcohol dehydrogenase (AdhA), amylase (amy), amyloglucosidase (AG- from the glaA gene), acetamidase (amdS) and glyceraldehyde-3-phosphate dehydrogenase (gpd) promoters.

Examples of strong yeast promoters which may be used include those obtainable from the genes for alcohol dehydrogenase, glyceraldehyde-3-phosphate dehydrogenase, lactase, 3-phosphoglycerate kinase, plasma membrane ATPase (PMA1) and triosephosphate isomerase.

Examples of strong bacterial promoters which may be used include the amylase and SPo2 promoters as well as promoters from extracellular protease genes.

Promoters suitable for plant cells which may be used include napaline synthase (nos), octopine synthase (ocs), mannopine synthase (mas), ribulose small subunit (rubisco ssu), histone, rice actin, phaseolin, cauliflower mosaic virus (CMV) 35S and 19S and circovirus promoters.

The vector may further include sequences flanking the polynucleotide giving rise to RNA which comprise sequences homologous to ones from eukaryotic genomic sequences, preferably fungal genomic sequences, or yeast genomic sequences. This will allow the introduction of the polynucleotides of the invention into the genome of fungi or yeasts by homologous recombination. In particular, a plasmid vector comprising the expression cassette flanked by fungal sequences can be used to prepare a vector suitable for delivering the polynucleotides of the invention to a fungal cell. Transformation techniques using these fungal vectors are known to those skilled in the art.

The vector may contain a polynucleotide of the invention oriented in an antisense direction to provide for the production of antisense RNA. This may be used to reduce, if desirable, the levels of expression of the polypeptide.

Host Cells and Expression

In a further aspect the invention provides a process for preparing a polypeptide of the invention which comprises cultivating a host cell transformed or transfected with an expression vector as described above under conditions suitable for expression by the vector of a coding sequence encoding the polypeptide, and recovering the expressed polypeptide. Polynucleotides of the invention can be incorporated into a recombinant replicable vector, such as an expression vector. The vector may be used to replicate the nucleic acid in a compatible host cell. Thus in a further embodiment, the invention provides a method of making a polynucleotide of the invention by introducing a polynucleotide of the invention into a replicable vector, introducing the vector into a compatible host cell, and growing the host cell under conditions which bring about the replication of the vector. Suitable host cells include bacteria such as E. coli, yeast, mammalian cell lines and other eukaryotic cell lines, for example insect cells such as Sf9 cells and (e.g. filamentous) fungal cells.

Preferably the polypeptide is produced as a secreted protein in which case the DNA sequence encoding a mature form of the polypeptide in the expression construct may be operably linked to a DNA sequence encoding a signal sequence. In the case where the gene encoding the secreted protein has in the wild type strain a signal sequence preferably the signal sequence used will be native (homologous) to the DNA sequence encoding the polypeptide. Alternatively the signal sequence is foreign (heterologous) to the DNA sequence encoding the polypeptide, in which case the signal sequence is preferably endogenous to the host cell in which the DNA sequence is expressed. Examples of suitable signal sequences for yeast host cells are the signal sequences derived from yeast MFalpha genes. Similarly, a suitable signal sequence for filamentous fungal host cells is e.g. a signal sequence derived from a filamentous fungal amyloglucosidase (AG) gene, e.g. the A.niger glaA gene. This signal sequence may be used in combination with the amyloglucosidase (also called (gluco) amylase) promoter itself, as well as in combination with other promoters. Hybrid signal sequences may also be used within the context of the present invention.

Preferred heterologous secretion leader sequences are those originating from the fungal amyloglucosidase (AG) gene (glaA—both 18 and 24 amino acid versions e.g. from Aspergillus), the MFalpha gene (yeasts e.g. Saccharomyces and Kluyveromyces) or the alpha-amylase gene (Bacillus).

The vectors may be transformed or transfected into a suitable host cell as described above to provide for expression of a polypeptide of the invention. This process may comprise culturing a host cell transformed with an expression vector as described above under conditions suitable for expression of the polypeptide, and optionally recovering the expressed polypeptide.

A further aspect of the invention thus provides host cells transformed or transfected with or comprising a polynucleotide or vector of the invention. Preferably the polynucleotide is carried in a vector which allows the replication and expression of the polynucleotide. The cells will be chosen to be compatible with the said vector and may for example be prokaryotic (for example bacterial), or eukaryotic fungal, yeast or plant cells.

The invention encompasses processes for the production of a polypeptide of the invention by means of recombinant expression of a DNA sequence encoding the polypeptide. For this purpose the DNA sequence of the invention can be used for gene amplification and/or exchange of expression signals, such as promoters, secretion signal sequences, in order to allow economic production of the polypeptide in a suitable homologous or heterologous host cell. A homologous host cell is herein defined as a host cell which is of the same species or which is a variant within the same species as the species from which the DNA sequence is derived.

Suitable host cells are preferably prokaryotic microorganisms such as bacteria, or more preferably eukaryotic organisms, for example fungi, such as yeasts or filamentous fungi, or plant cells. In general, yeast cells are preferred over filamentous fungal cells because they are easier to manipulate. However, some proteins are either poorly secreted from yeasts, or in some cases are not processed properly (e.g. hyperglycosylation in yeast). In these instances, a filamentous fungal host organism should be selected.

Bacteria from the genus Bacillus are very suitable as heterologous hosts because of their capability to secrete proteins into the culture medium. Other bacteria suitable as hosts are those from the genera Streptomyces and Pseudomonas. A preferred yeast host cell for the expression of the DNA sequence encoding the polypeptide is one of the genus Saccharomyces, Kluyveromyces, Hansenula, Pichia, Yarrowia, or Schizosaccharomyces. More preferably, a yeast host cell is selected from the group consisting of the species Saccharomyces cerevisiae, Kluyveromyces lactis (also known as Kluyveromyces marxianus var. lactis), Hansenula polymorpha, Pichia pastoris, Yarrowia lipolytica, and Schizosaccharomyces pombe.

Most preferred for the expression of the DNA sequence encoding the polypeptide are, however, filamentous fungal host cells. Preferred filamentous fungal host cells are selected from the group consisting of the genera Aspergillus, Trichoderma, Fusarium, Disporotrichum, Penicillium, Acremonium, Neurospora, Thermoascus, Myceliophtora, Sporotrichum, Thielavia, and Talaromyces. More preferably a filamentous fungal host cell is of the species Aspergillus oyzae, Aspergillus sojae or Aspergillus nidulans or is of a species from the Aspergillus niger Group (as defined by Raper and Fennell, The Genus Aspergillus, The Williams & Wilkins Company, Baltimore, pp 293-344, 1965). These include but are not limited to Aspergillus niger, Aspergillus awamori, Aspergillus tubigensis, Aspergillus aculeatus, Aspergillus foetidus, Aspergillus nidulans, Aspergillus japonicus, Aspergillus oryzae and Aspergillus ficuum, and also those of the species Trichoderma reesei, Fusarium graminearum, Penicillium chrysogenum, Acremonium alabamense, Neurospora crassa, Myceliophtora thermophilum, Sporotrichum cellulophilum, Disporotrichum dimorphosporum and Thielavia terrestris.

Examples of preferred expression hosts within the scope of the present invention are fungi such as Aspergillus species (in particular those described in EP-A-184,438 and EP-A-284,603) and Trichoderma species; bacteria such as Bacillus species (in particular those described in EP-A-134,048 and EP-A-253,455), especially Bacillus subtilis, Bacillus licheniformis, Bacillus amyloliquefaciens, Pseudomonas species; and yeasts such as Kluyveromyces species (in particular those described in EP-A-096,430 such as Kluyveromyces lactis and in EP-A-301,670) and Saccharomyces species, such as Saccharomyces cerevisiae.

Host cells according to the invention include plant cells, and the invention therefore extends to transgenic organisms, such as plants and parts thereof, which contain one or more cells of the invention. The cells may heterologously express the polypeptide of the invention or may heterologously contain one or more of the polynucleotides of the invention. The transgenic (or genetically modified) plant may therefore have inserted (typically stably) into its genome a sequence encoding the polypeptides of the invention. The transformation of plant cells can be performed using known techniques, for example using a Ti or a Ri plasmid from Agrobacterium tumefaciens. The plasmid (or vector) may thus contain sequences necessary to infect a plant, and derivatives of the Ti and/or Ri plasmids may be employed.

The host cell may overexpress the polypeptide, and techniques for engineering over-expression are well known and can be used in the present invention. The host may thus have two or more copies of the polynucleotide.

Alternatively, direct infection of a part of a plant, such as a leaf, root or stem can be effected. In this technique the plant to be infected can be wounded, for example by cutting the plant with a razor, puncturing the plant with a needle or rubbing the plant with an abrasive. The wound is then innoculated with the Agrobacterium. The plant or plant part can then be grown on a suitable culture medium and allowed to develop into a mature plant. Regeneration of transformed cells into genetically modified plants can be achieved by using known techniques, for example by selecting transformed shoots using an antibiotic and by sub-culturing the shoots on a medium containing the appropriate nutrients, plant hormones and the like.

Culture of Host Cells and Recombinant Production

The invention also includes cells that have been modified to express the peptidyl arginine deiminase or a variant thereof. Such cells include transient, or preferably stably modified higher eukaryotic cell lines, such as mammalian cells or insect cells, lower eukaryotic cells, such as yeast and filamentous fungal cells or prokaryotic cells such as bacterial cells.

It is also possible for the polypeptides of the invention to be transiently expressed in a cell line or on a membrane, such as for example in a baculovirus expression system. Such systems, which are adapted to express the proteins according to the invention, are also included within the scope of the present invention.

According to the present invention, the production of the polypeptide of the invention can be effected by the culturing of microbial expression hosts, which have been transformed with one or more polynucleotides of the present invention, in a conventional nutrient fermentation medium.

The recombinant host cells according to the invention may be cultured using procedures known in the art. For each combination of a promoter and a host cell, culture conditions are available which are conducive to the expression the DNA sequence encoding the polypeptide. After reaching the desired cell density or titre of the polypeptide the culturing is ceased and the polypeptide is recovered using known procedures.

The fermentation medium can comprise a known culture medium containing a carbon source (e.g. glucose, maltose, molasses, etc.), a nitrogen source (e.g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.). Optionally, an inducer (dependent on the expression construct used) may be included or subsequently be added.

The selection of the appropriate medium may be based on the choice of expression host and/or based on the regulatory requirements of the expression construct. Suitable media are well-known to those skilled in the art. The medium may, if desired, contain additional components favoring the transformed expression hosts over other potentially contaminating microorganisms.

The fermentation may be performed over a period of from 0.5-30 days. Fermentation may be a batch, continuous or fed-batch process, at a suitable temperature in the range of between 0° C. and 45° C. and, for example, at a pH from 2 to 10. Preferred fermentation conditions include a temperature in the range of between 20° C. and 37° C. and/or a pH between 3 and 9. The appropriate conditions are usually selected based on the choice of the expression host and the protein to be expressed.

After fermentation, if necessary, the cells can be removed from the fermentation broth by means of centrifugation or filtration. After fermentation has stopped or after removal of the cells, the polypeptide of the invention may then be recovered and, if desired, purified and isolated by conventional means. The peptidyl arginine deiminase of the invention can be purified from fungal mycelium or from the culture broth into which the peptidyl arginine deiminase is released by the cultured fungal cells.

In a preferred embodiment the polypeptide produced from a fungus, more preferably from an Aspergillus, most preferably from Aspergillus niger.

Modifications

Polypeptides of the invention may be chemically modified, e.g. post-translationally modified. For example, they may be glycosylated (one or more times) or comprise modified amino acid residues. They may also be modified by the addition of histidine residues to assist their purification or by the addition of a signal sequence to promote secretion from the cell. The polypeptide may have amino- or carboxyl-terminal extensions, such as an amino-terminal methionine residue, a small linker peptide of up to about 20-25 residues, or a small extension that facilitates purification, such as a poly-histidine tract, an antigenic epitope or a binding domain.

A polypeptide of the invention may be labelled with a revealing label. The revealing label may be any suitable label which allows the polypeptide to be detected. Suitable labels include radioisotopes, e.g. ¹²⁵I, ³⁵S, enzymes, antibodies, polynucleotides and linkers such as biotin.

The polypeptides may be modified to include non-naturally occurring amino acids or to increase the stability of the polypeptide. When the proteins or peptides are produced by synthetic means, such amino acids may be introduced during production. The proteins or peptides may also be modified following either synthetic or recombinant production.

The polypeptides of the invention may also be produced using D-amino acids. In such cases the amino acids will be linked in reverse sequence in the C to N orientation. This is conventional in the art for producing such proteins or peptides.

A number of side chain modifications are known in the art and may be made to the side chains of the proteins or peptides of the present invention. Such modifications include, for example, modifications of amino acids by reductive alkylation by reaction with an aldehyde followed by reduction with NaBH₄, amidination with methylacetimidate or acylation with acetic anhydride.

The sequences provided by the present invention may also be used as starting materials for the construction of “second generation” enzymes. “Second generation” peptidyl arginine deiminases are peptidyl arginine deiminases, altered by mutagenesis techniques (e.g. site-directed mutagenesis or gene shuffling techniques), which have properties that differ from those of wild-type peptidyl arginine deiminase or recombinant peptidyl arginine deiminase such as those produced by the present invention. For example, their temperature or pH optimum, specific activity, substrate affinity or thermostability may be altered so as to be better suited for use in a particular process.

Amino acids essential to the activity of the peptidyl arginine deiminase of the invention, and therefore preferably subject to substitution, may be identified according to procedures known in the art, such as site-directed mutagenesis or alanine-scanning mutagenesis. In the latter technique mutations are introduced at every residue in the molecule, and the resultant mutant molecules are tested for biological activity (e.g. peptidyl arginine deiminase activity) to identify amino acid residues that are critical to the activity of the molecule. Sites of enzyme-substrate interaction can also be determined by analysis of crystal structure as determined by such techniques as nuclear magnetic resonance, crystallography or photo-affinity labelling.

Gene shuffling techniques provide a random way to introduce mutations in a polynucleotide sequence. After expression the isolates with the best properties are re-isolated, combined and shuffled again to increase the genetic diversity. By repeating this procedure a number of times, genes that code for fastly improved proteins can be isolated. Preferably the gene shuffling procedure is started with a family of genes that code for proteins with a similar function. The family of polynucleotide sequences provided with this invention would be well suited for gene shuffling to improve the properties of secreted peptidyl arginine deiminases.

Alternatively classical random mutagenesis techniques and selection, such as mutagenesis with NTG treatment or UV mutagenesis, can be used to improve the properties of a protein. Mutagenesis can be performed directly on isolated DNA, or on cells transformed with the DNA of interest. Alternatively, mutations can be introduced in isolated DNA by a number of techniques that are known to the person skilled in the art. Examples of these methods are error-prone PCR, amplification of plasmid DNA in a repear-deficient host cell, etc.

The use of yeast and filamentous fungal host cells is expected to provide for post-translational modifications (e.g. proteolytic processing, myristilation, glycosylation, truncation, and tyrosine, serine or threonine phosphorylation) as may be needed to confer optimal biological activity on recombinant expression products of the invention.

Preparations

Polypeptides of the invention may be in an isolated form. It will be understood that the polypeptide may be mixed with carriers or diluents which will not interfere with the intended purpose of the polypeptide and still be regarded as isolated. A polypeptide of the invention may also be in a substantially purified form, in which case it will generally comprise the polypeptide in a preparation in which more than 70%, e.g. more than 80%, 90%, 95%, 98% or 99% of the proteins in the preparation is a polypeptide of the invention.

Polypeptides of the invention may be provided in a form such that they are outside their natural cellular environment. Thus, they may be substantially isolated or purified, as discussed above, or in a cell in which they do not occur in nature, for example a cell of other fungal species, animals, plants or bacteria.

Removal or Reduction of Peptidyl Arginine Deiminase Activity

The present invention also relates to methods for producing a mutant cell of a parent cell, which comprises disrupting or deleting the endogenous nucleic acid sequence encoding the polypeptide or a control sequence thereof, which results in the mutant cell producing less of the polypeptide than the parent cell.

The construction of strains which have reduced peptidyl arginine deiminase activity may be conveniently accomplished by modification or inactivation of a nucleic acid sequence necessary for expression of the peptidyl arginine deiminase in the cell. The nucleic acid sequence to be modified or inactivated may be, for example, a nucleic acid sequence encoding the polypeptide or a part thereof essential for exhibiting peptidyl arginine deiminase activity, or the nucleic acid sequence may have a regulatory function required for the expression of the polypeptide from the coding sequence of the nucleic acid sequence. An example of such a regulatory or control sequence may be a promoter sequence or a functional part thereof, i.e., a part which is sufficient for affecting expression of the polypeptide. Other control sequences for possible modification include, but are not limited to, a leader sequence, a polyadenylation sequence, a propeptide sequence, a signal sequence, and a termination sequence.

Modification or inactivation of the nucleic acid sequence may be performed by subjecting the cell to mutagenesis and selecting cells in which the peptidyl arginine deiminase producing capability has been reduced or eliminated. The mutagenesis, which may be specific or random, may be performed, for example, by use of a suitable physical or chemical mutagenizing agent, by use of a suitable oligonucleotide, or by subjecting the DNA sequence to PCR mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (NTG), O-methyl hydroxylamine, nitrous acid, ethyl methane sulphonate (EMS), sodium bisulphite, formic acid, and nucleotide analogues.

When such agents are used, the mutagenesis is typically performed by incubating the cell to be mutagenized in the presence of the mutagenizing agent of choice under suitable conditions, and selecting for cells exhibiting reduced or no expression of peptidyl arginine deiminase activity.

Modification or inactivation of production of a polypeptide of the present invention may be accomplished by introduction, substitution, or removal of one or more nucleotides in the nucleic acid sequence encoding the polypeptide or a regulatory element required for the transcription or translation thereof. For example, nucleotides may be inserted or removed so as to result in the introduction of a stop codon, the removal of the start codon, or a change of the open reading frame. Such modification or inactivation may be accomplished by site-directed mutagenesis or PCR mutagenesis in accordance with methods known in the art.

Although, in principle, the modification may be performed in vivo, i.e., directly on the cell expressing the nucleic acid sequence to be modified, it is preferred that the modification be performed in vitro as exemplified below.

An example of a convenient way to inactivate or reduce production of the peptidyl arginine deiminase by a host cell of choice is based on techniques of gene replacement or gene interruption. For example, in the gene interruption method, a nucleic acid sequence corresponding to the endogenous gene or gene fragment of interest is mutagenized in vitro to produce a defective nucleic acid sequence which is then transformed into the host cell to produce a defective gene. By homologous recombination, the defective nucleic acid sequence replaces the endogenous gene or gene fragment. Preferably the defective gene or gene fragment also encodes a marker which may be used to select for transformants in which the gene encoding the polypeptide has been modified or destroyed.

Alternatively, modification or inactivation of the nucleic acid sequence encoding a polypeptide of the present invention may be achieved by established anti-sense techniques using a nucleotide sequence complementary to the polypeptide encoding sequence. More specifically, production of the polypeptide by a cell may be reduced or eliminated by introducing a nucleotide sequence complementary to the nucleic acid sequence encoding the polypeptide. The antisense polynucleotide will then typically be transcribed in the cell and will be capable of hybridizing to the mRNA encoding the peptidyl arginine deiminase. Under conditions allowing the complementary antisense nucleotide sequence to hybridize to the mRNA, the amount of the peptidyl arginine deiminase produced in the cell will be reduced or eliminated.

It is preferred that the cell to be modified in accordance with the methods of the present invention is of microbial origin, for example, a fungal strain which is suitable for the production of desired protein products, either homologous or heterologous to the cell.

The present invention further relates to a mutant cell of a parent cell which comprises a disruption or deletion of the endogenous nucleic acid sequence encoding the polypeptide or a control sequence thereof, which results in the mutant cell producing less of the polypeptide than the parent cell.

The polypeptide-deficient mutant cells so created are particularly useful as host cells for the expression of homologous and/or heterologous polypeptides. Therefore, the present invention further relates to methods for producing a homologous or heterologous polypeptide comprising (a) culturing the mutant cell under conditions conducive for production of the polypeptide; and (b) recovering the polypeptide. In the present context, the term “heterologous polypeptides” is defined herein as polypeptides which are not native to the host cell, a native protein in which modifications have been made to alter the native sequence, or a native protein whose expression is quantitatively altered as a result of a manipulation of the host cell by recombinant DNA techniques.

In a still further aspect, the present invention provides a method for producing a protein product essentially free of peptidyl arginine deiminase activity by fermentation of a cell which produces both an peptidyl arginine deiminase polypeptide of the present invention as well as the protein product of interest. The method comprises adding an effective amount of an agent capable of inhibiting peptidyl arginine deiminase activity to the fermentation broth either during or after the fermentation has been completed, recovering the product of interest from the fermentation broth, and optionally subjecting the recovered product to further purification. Alternatively, after cultivation the resultant culture broth can be subjected to a pH or temperature treatment so as to reduce the peptidyl arginine deiminase activity substantially, and allow recovery of the product from the culture broth. The combined pH or temperature treatment may be performed on an protein preparation recovered from the culture broth.

The methods of the present invention for producing an essentially peptidyl arginine deiminase-free product is of particular interest in the production of eukaryotic polypeptides, in particular in the production of fungal proteins such as enzymes. The peptidyl arginine deiminase-deficient cells may also be used to express heterologous proteins of interest for the food industry, or of pharmaceutical interest.

Preferred sources for the peptidyl arginine deiminase are obtained by cloning a microbial gene encoding a peptidyl arginine deiminase into a microbial host organism, More preferred sources for the peptidyl arginine deiminase are obtained by cloning an Fusarium-derived gene encoding a peptidyl arginine deiminase into a host belonging to the genus of Aspergillus capable of overexpressing the peptidyl arginine deiminase gene. In general homologeous host organisms are preferred for overexpression. Homologeous is meant here being of the same species.

### List of Sequences

SEQ ID NO: 1 synthetic DNA
SEQ ID NO: 2 synthetic DNA
SEQ ID NO: 3 DNA of Fusarium graminearum
SEQ ID NO: 4 DNA of Fusarium graminearum
SEQ ID NO: 5 DNA of Fusarium graminearum
SEQ ID NO: 6 amino acid sequence of Fusarium graminearum
SEQ ID NO: 7 amino acid sequence of Fusarium graminearum
SEQ ID NO: 8 amino acid sequence of Chaetomium globosum
SEQ ID NO: 9 amino acid sequence of Streptomyces scabies
SEQ ID NO: 10 amino acid sequence of Streptomyces scabies
SEQ ID NO: 11 PAD consensus amino acid sequence
SEQ ID NO: 12 artificial amino acid sequence
SEQ ID NO: 13 amino acid sequence of Streptomyces clavuligerus
SEQ ID NO, 14 amino acid sequence of Phaeosphaeria nodorum

### Legends to the Figures

FIG. 1: Cloning of Fusarium PAD,

FIG. 2: Alignment of secreted PAD proteins.

FIG. 3: Construction of expression vector pGBFINPAD.

FIG. 4; Comparing protein concentrations of BSA and PAD on SDS-PAGE, Staining was performed using Simply Blue Safe Stain (Collodial Coomassie G250).
Lanes 1-6 BSA, lanes 7-10 PAD.

### Materials & Methods

### Degree of Hydrolysis

The Degree of Hydrolysis (DH) of the various protolytic mixtures used was measured using a rapid OPA test (Nielsen, P. M.-, Petersen, D.; Dambmann, C. Improved method for determining food protein degree of hydrolysis. Journal of Food Science 2001, 66, 642-646).

### PAD Assays

PAD-like activity was monitored in two different ways. In screening assays the Sigma Quality Control Test Procedure as provided by Sigma-Aldrich for this enzyme (p1584; from rabbit skeletal muscle) was used. This chromogenic method is based on the use of N-alpha-benzoyl-L-arginine ethyl ester hydrochloride (BAEE; Takahara et al. (1986) Journal of Biochemistry, 99, 1477-1424).
In another assay the activity of the enzyme is measured by classical amino acid analysis by measuring the conversion of free or peptide bound arginine into citrulline. This method is specified under “Amino acid analysis”.

### Amino Acid Analysis

Amino acid analyses were carried out according to the PicoTag method as specified in the operators manual of the Amino Acid Analysis System of Waters (Milford Mass., USA). To that end samples were dried and directly derivatised using phenylisothiocyanate. The positions of citrulline and ornithine in the chromatogram were established by test runs in which the pure compounds (Sigma) were derivatised and subjected to chromatography. The derivatised amino acids present were quantitated using HPLC methods. Incubation mixtures in which proteins or peptides were used as the substrate for PAD were first acid hydrolysed according to the operators manual of the Amino Acid Analysis System of Waters (Milford Mass., USA), then derivatised and separated and quantitated. In incubation mixtures containing free arginine only, acid hydrolysis was optional. As exemplified in Example 6, an acid hydrolysis partially converts citrulline into ornithine. To calculate the total amount of citrulline formed from arginine, the levels of citrulline and ornithine were added up. During the acid hydrolysis Trp and Cys are destroyed, therefore these amino acids were omitted in further calculations. Furthermore, Gln and Asn residues are converted into Glu and Asp during acid hydrolysis so that the values for Glu and Gln, and for Asp and Asn are also added up to allow comparison with the data obtained before acid hydrolysis.

### SDS-PAGE

All materials used for SDS-PAGE and staining were purchased from Invitrogen (Carlsbad, Calif., U.S.). Samples were prepared using SDS buffer according to manufacturers instructions and separated on 12% Bis-Tris gels using MES-SDS buffer system according to manufacturers instructions. Staining was performed using Simply Blue Safe Stain (Collodial Coomassie G250).

### LC/MS/MS Analysis

In the analysis of peptide QPRPFPFPRPR after PAD incubation, an HPLC using an ion trap mass spectrometer (Thermoquest®, Breda, the Netherlands) coupled to a P4000 pump (Thermoquest®, Breda, the Netherlands) was used. Peptides formed were separated using a Inertsil 3 ODS 3, 3 μm, 150* 2.1 mm (Varian Belgium, Belgium) column in combination with a gradient of 0.1% formic acid in Milli Q water (Millipore, Bedford, Mass., USA; Solution A) and 0.1% formic acid in acetonitrile (Solution B) for elution. The gradient started at 100% of Solution A, kept here for 5 minutes, increasing linear to 5% B in 10 minutes, followed by a linear increasing to 45% of solution B in 30 minutes and immediately going to the beginning conditions, and kept there for another 15 minutes for stabilization. The injection volume used was 50 microliters, the flow rate was 200 microliter per minute and the column temperature was maintained at 55° C. The protein concentration of the injected sample was approx. 50 micrograms/milliliter. The reaction and reaction rate of the different arginine residues in peptide QPRPFPFPRPR after incubation was followed in time by dedicated MS/MS for the peptides of interest, using optimal collision energy of about 30%. Prior to LC/MS/MS the incubation mixtures were centrifuged at ambient temperature and 13000 rpm for 10 minutes, filtered through a 0.22 μm filter and the supernatant was diluted 1:100 with MilliQ water.

### Cloning Techniques

Standard molecular cloning techniques such as isolation and purification of nucleic acids, electrophoresis of nucleic acids, enzymatic modification, cleavage and/or amplification of nucleic acids, transformation of E. coli, etc., were performed according to Sambrook et al (Sambrook, J., Russell, D. W. (2001): Molecular cloning; a laboratory manual (third edition). Cold Spring Harbour laboratory press, Cold Spring Harbour, N.Y.), or to the supplier's specifications. Invitrogen (Breda, the Netherlands) supplied synthetic oligonucleotides. DNA sequence analyses were performed at BaseClear (Leiden, the Netherlands).

### EXAMPLES

### Example 1

### Fusarium Strains can Secrete a PAD-Like Activity

A large collection of moulds was screened with the aim of identifying a secreted PAD-like activity. To that end strains were pre-grown for 4-5 days at 30 degrees C. on Potato Dextrose Broth (PDB; Difco). Then, cultures were harvested by centrifugation, washed with distilled water and transferred to a minimal medium enriched with rice protein hydrolysate. Rice protein is relatively rich in arginine residues and to increase its water solubility, the rice protein (Remy Industries, Leuven, Belgium) was pre-incubated with Alcalase (NOVO, Bagsvaerd, Denmark) at pH 7.5 to obtain a hydrolysate with a DH of approx 15. The minimal growth medium as used contained 0.52 g KCl, 1.52 g KH₂PO₄, 1.3 ml 4M KOH, 0.52 g MgSO₄.7H₂O, 22 mg ZnSO₄.7H₂O, 11 mg H₃BO₃, 5 mg FeSO₄.7H₂O, 1.7 mg CoCl₂.6H₂O, 1.6 mg CuSO₄.5H₂O, 5 mg MnCl₂.4H₂O, 1.5 mg Na₂MoO₄.2H₂O, 50 mg EDTA, 40 g glucose and 5 g of hydrolyzed rice protein per liter. After growing the fungi for another 2 days in the latter medium, the cultures were again centrifuged and samples of the clear supernatants were frozen. To determine whether a secreted PAD-like activity was present, supernatant samples were subjected to the colorimetric Sigma Quality Control Test Procedure as described in the Materials & Methods section. In this test 0.2 ml of the undiluted supernatant was added to 0.6 ml of the mixed reagents. Enzyme incubation took place for 5 hours at 45 degrees C. According to the results obtained, the supernatants of some, but not all Fusarium strains showed some discoloration. Supernatants leading to a discoloration, were obtained from Fusarium graminearum strains CBS166.57, CBS316.73, CBS11063, CBS18432 and CBS792.70 as obtained from CBS, Utrecht, The Netherlands. The supernatants of several other Fusarium strains and other fungi, e.g. Fusarium graminearum IMI145425 (CABI, Wallingford, UK), did not generate a color. From the results obtained, we concluded that some Fusarium strains secrete a PAD-like activity. To our knowledge this is the first report of the secretion of a PAD from any micro-organism.

### Example 2

### Identifying PAD Encoding Genes in a Fusarium Genome Sequence

Knowing that some Fusarium strains can secrete a PAD-like activity, the genome sequence of the genes encoding these PAD's were isolated and analyzed. To do this, Fusarium graminearum strains CBS166.57, CBS316.73, CBS11063, CBS18432 and CBS792.70 were grown for 3 days at 30 degrees Celsius in PDB (Potato dextrose broth, Difco) and chromosomal DNA was isolated from the mycelium using the Q-Biogene kit (catalog nr. 6540-600; Omnilabo International BV, Breda, the Netherlands), using the instructions of the supplier. This chromosomal DNA was used for the amplification of the coding sequence of the PAD genes using PCP.

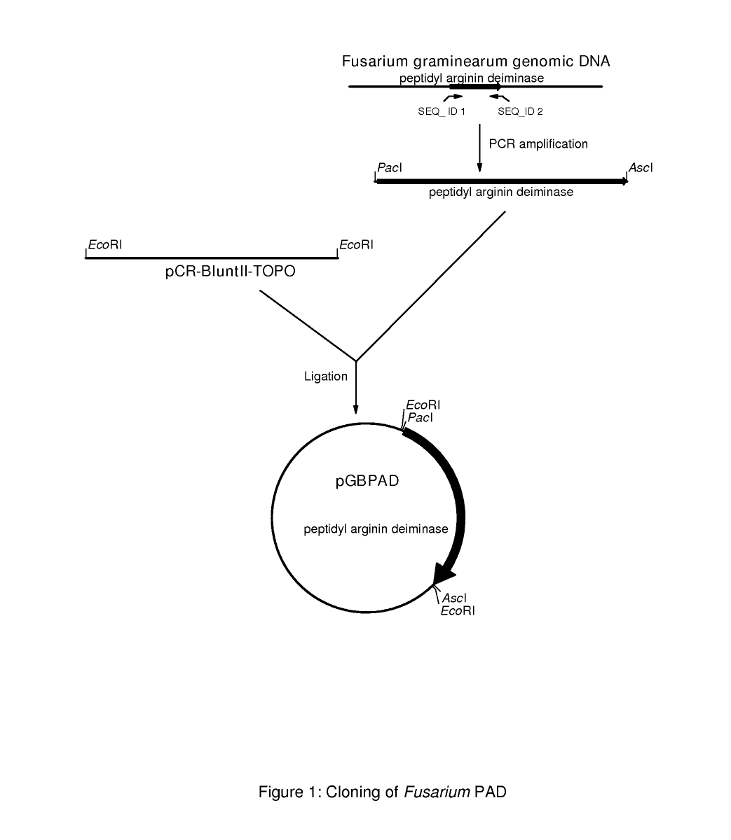

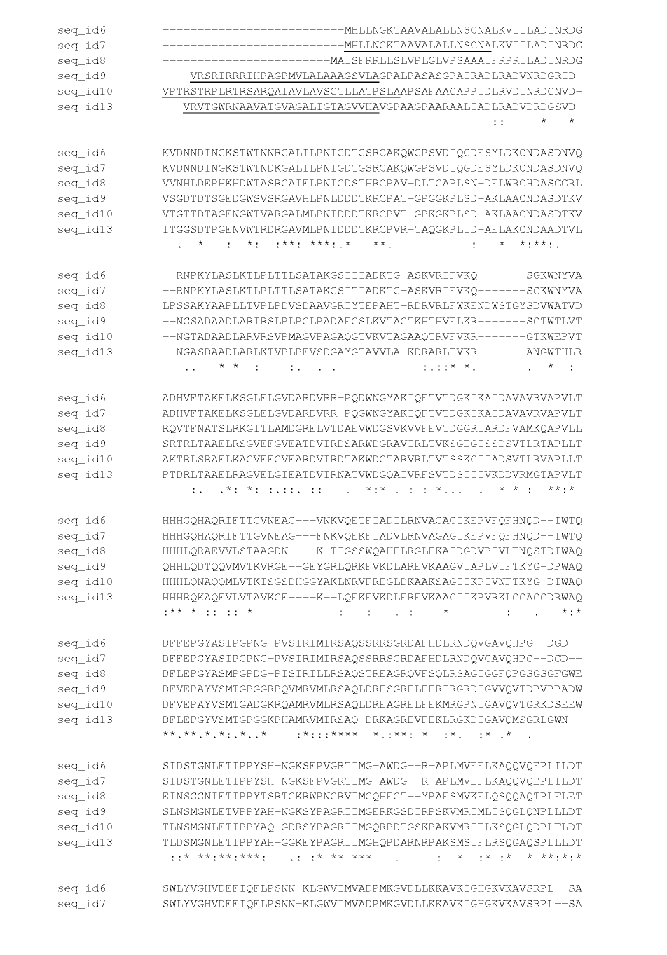

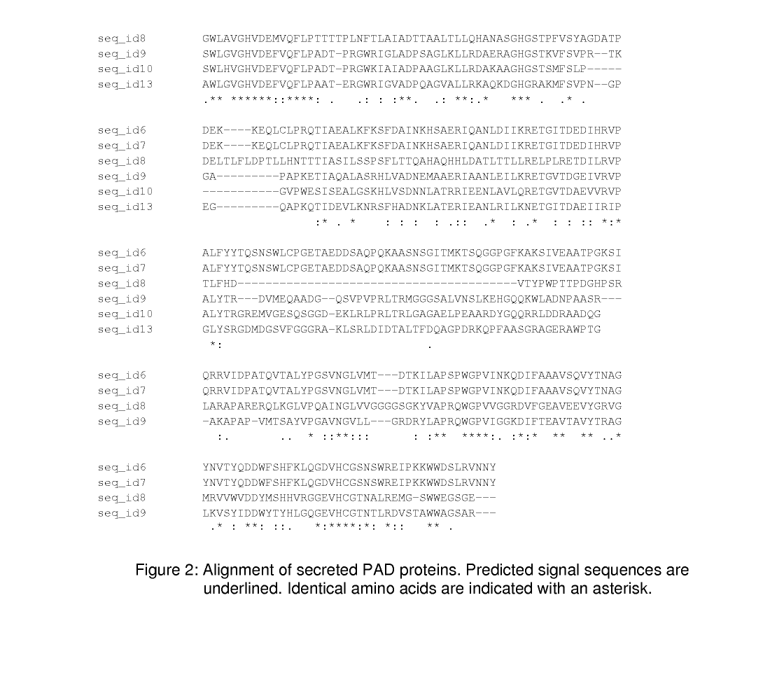

To specifically amplify the PAD gene from the chromosomal DNA of Fusarium graminearum strains CBS66.57, CBS316.73, CBS11063, CBS18432 and CBS799.70, two PCR primers were designed. Primer sequences were partly obtained from a sequence that was found in the genomic DNA of Gibberella zeae PH-1 and annotated as hypothetical protein (UNIPROT Q4IIR5_GIBZE). We found that this sequence has homology with PAD sequences of higher eukaryotes. The first primer contained 24 nucleotides PAD coding sequences starting at the ATG start codon with an upstream 12 bps leader sequence and a Pacl restriction site (SEQ ID NO: 1). The second primer contained 29 nucleotides complementary to the PAD coding sequences with an Ascl restriction site immediately downstream the CTA stop codon (SEQ ID NO: 2). Using these primers we were able to amplify a 2 kb sized fragment with chromosomal DNA from Fusarium graminearum strains CBS166.57, CBS316.73, CBS11063, CBS18432 and CBS792.70 as template. In all these cases the amplified fragment was of the same size. The thus obtained 2 kb sized fragments were purified and ligated into the pCR-BluntII-TOPO vector (Invitrogen) resulting in plasmids of the pGBPAD series (see FIG. 1). PCR amplified sequences were analyzed by sequence analysis. Interestingly, we were not able to amplify a genomic DNA fragment of this size from i.e. Fusarium graminearum IMI145425, suggesting that the presence of the 2 kb fragment correlates with the production of a secreted PAD activity in Fusarium graminearum.
The genomic sequences of the PAD coding region of the Fusarium graminearum strains CBS166.57 and CBS316.73, is depicted in SEQ ID NO: 3 and SEQ ID NO: 4 respectively. The cDNA sequence of the coding region was generated from the mRNA isolated from a strain over expressing the PAD of Fusarium graminearum CBS166.57 (see Example 3). This cDNA was sequenced and is depicted in SEQ ID NO: 5. The deduced protein sequences of the PAD's encoded by Fusarium graminearum strains CBS166.57 and CBS316.73 is depicted in SEQ ID NO: 6 and SEQ ID NO: 7 respectively.

The genomic DNA sequence of the PAD of Fusarium graminearum CBS166.57 differs at 19 positions from the PAD genomic DNA sequence from Fusarium graminearum CBS316.73. For the deduced protein sequence this means that 7 amino acids are different and the two PAD's from Fusarium graminearum strains CBS166.57 and CBS316.73 are 98.0% identical. Interestingly, both deduced protein sequences contain a sequence signal at the amino-terminus of the protein. These sequences were compared to the protein and DNA databases using the program BlastP (Altschul et al., 1997, Nucleic Acids Research 25: 3389-3402) with matrix Blosum 62 and an expected threshold of 10. We found that both the DNA and the deduced protein sequence of the PAD of Fusarium graminearum CBS166.57 was identical to the sequence that was found in the genomic DNA of Gibberella zeae PH-1 and annotated as hypothetical protein (UNIPROT Q4IIR5_GIBZE). The PAD from Fusarium also had significant homology to the PAD's of higher eukaryotes, with the essential difference that the fungal protein sequences contain a signal sequence and are therefore most likely actively secreted from the cell, and the PAD's from higher eukaryotes do not have such a signal sequence and are therefore not actively secreted. The Fusarium PAD did not show any homology with the PAD from Porphyromonas.

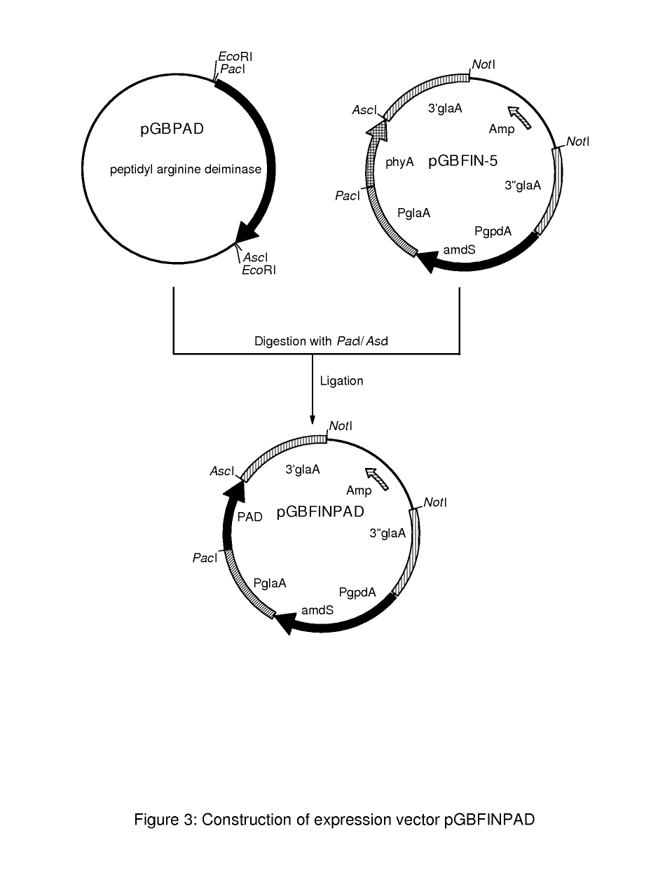

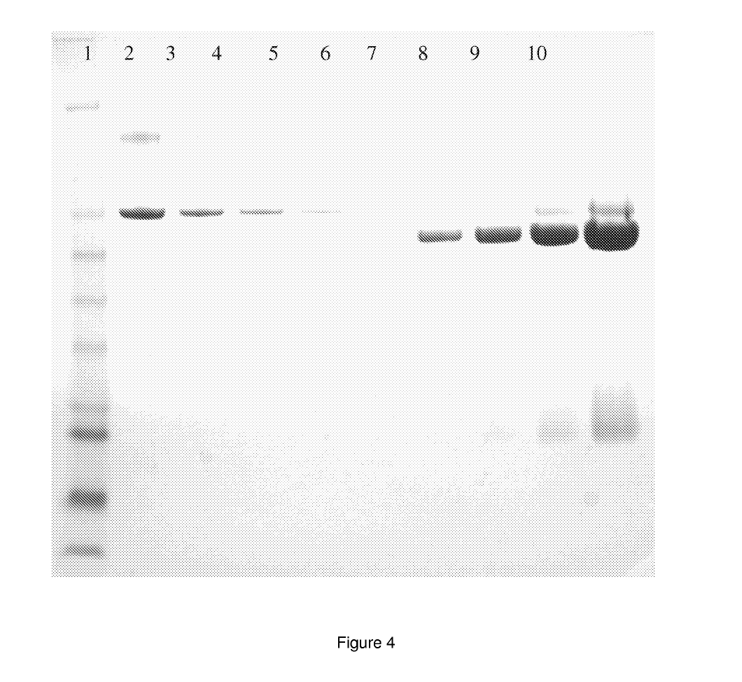

Additionally, homology was found between the Fusarium PAD's and a sequence from the genome of Chaetomium globosum CBS 148.51 (http:/www.broad.mit.edu/annotation/genome/chaetomium_globosum/Home.html; CHGG_{—}01998.1), another ascomycetes fungus (like Fusarium) but belonging to a different order (Sordariales instead of the Hypocreales). Homology between Fusarium and Chaetomium PAD sequences is 253 amino acids over 660 amino acids (38% identical). Also here, the predicted protein is annotated as hypothetical protein (UNIPROT Q2HCQ6_CHAGB). Additionally, the annotation of the protein sequence of the PAD homologue in this fungus is probably incorrect. Homology of the Chaetomium protein to the known PAD's is only present over the first 600 amino acids of the protein, while the Chaetomium protein is annotated as a protein of 1000 amino acids. We therefore think that the annotation of the Chaetomium protein in UNIPROT Q2HCQ6_CHAGB is incorrect. The correct protein sequence of Chaetomium globosum PAD is depicted in SEQ ID NO: 8 and alignment is shown in FIG. 3. When this sequence is inspected more closely using the program SignalP (http://www.cbs.dtu.dk/services/SignalP/), a signal sequence can be detected at the amino-terminus of the protein (see FIG. 3), meaning that also this protein is most likely secreted from the fungus, like we have found for the PAD's from the Fusarium strains.

Another homologue of the Fusarium PAD's could be found in the genome of Phaeosphaeria nodorum SN15, also an ascomycetes fungus (like Fusarium and Chaetomium) but belonging even to a different class (Dithideomycetes instead of the Sordariomycetes). The protein encoded by this gene is depicted in SEQ ID NO: 14. Homology between Fusarium and Phaeosphaeria PAD sequences is 231 amino acids over 629 amino acids (36% identical). Also here, the predicted protein is annotated as hypothetical protein (hypothetical protein SNOG_{—}13103; Genebank EAT79430), and a signal sequence can be recognized. The data presented here suggest that secreted PAD's can be present throughout the fungal kingdom, but occur very infrequently. Using the methods described here within one will be able to specifically recognize and isolate secreted PAD's from fungi.

When the databases were inspected further for possible homologues of the Fusarium PAD, we could find two additional sequences in the genome of Streptomyces scabies, the causative agent of potato scab (http://www.sanger.ac.uk/Projects/S_scabies/). Protein sequences of these PAD's are depicted in SEQ ID NO: 9 and SEQ ID NO: 10. Also in these genes it is possible to suspect a signal sequence at the amino terminus of the protein, so also these proteins might be secreted. The carboxyl-terminus of the protein depicted in SEQ ID NO. 10 is missing, since this gene is located at the end of a contig, and the 3′-terminus of the coding region is not yet sequenced.

When the genome of Streptomyces clavuligerus ATCC27064 was sequenced and analysed, we could detect another homologue of PAD, again also containing a signal sequence as detected using the SignalP program. The amino acid sequence is depicted in SEQ ID NO: 13. The protein is short at the carboxy terminus since the gene was located at the end of a sequencing contig, and therefore the 3′-end was missing.

When comparing these PAD sequences from micro-organisms with the PAD's of higher eukaryotes, there is a very homologous region detectable between these sequences. This consensus sequence is WLxVGHVDE and depicted in SEQ ID NO: 11. Using this consensus sequence it is very well possible for those skilled in the art to identify and isolate the genes coding for PAD's from micro-organisms, using known cloning techniques. A possibility is to design oligonucleotide primers based on the back-translation of the sequence of SEQ ID NO: 11 into a nucleotide sequence with preferred codon usage from the organism in which one wants to identify a PAD gene, and using this oligonucleotide for hybridization to a gene library, or in a PCR primer on a reverse transcribed mRNA pool. Another possibility is to use the sequence of SEQ ID NO: 11 for a search in translated DNA sequences from a DNA databank using a program like Patscan (http://www-unix.mcs.anl.gov/compbio/PatScan/HTML/patscan.html). The genes that are identified using one of these methods can than be translated into a protein sequence using programs known to those skilled in the art, inspected for the presence of a signal sequence at their amino-terminus. For detecting a signal sequence one can use a program like SignalP (http:/www.cbs.dtu.dk/services/SignalP/). In this invention we have found that a protein sequence that contains both the consensus of SEQ ID NO: 11 and a predicted signal sequence is likely to be a secreted PAD. Looking for these combined properties gives a large advantage for the industrial production of such an enzyme.

### Example 3

### Over Expression of a Putative Fusarium Graminearum PAD by Aspergillus Niger

From the pGBPAD plasmid containing the genomic PAD gene from Fusarium graminearum CBS166.57, the Pacl/Ascl fragment comprising the PAD coding sequences was isolated and exchanged with the Pacl/Ascl phyA fragment in pGBFIN-5 (WO 99/32617). Resulting plasmid is the PAD expression vector named pGBFINPAD (see FIG. 3). The expression vector pGBFINPAD was linearized by digestion with Notl, which removes all E. coli derived sequences from the expression vector. The digested DNA was purified using phenol:chloroform:isoamylalcohol (24:23:1) extraction and precipitation with ethanol. These vectors were used to transform Aspergillus niger CBS513.88. An Aspergillus niger transformation procedure is extensively described in WO 98/46772. It is also described how to select for transformants on agar plates containing acetamide, and to select targeted multicopy integrants. Preferably, A. niger transformants containing multiple copies of the expression cassette are selected for further generation of sample material. For the pGBFIN PAD expression vector 30 A. niger transformants were purified; first by plating individual transformants on selective medium plates followed by plating a single colony on PDA plates. Spores of individual transformants were collected after growth for 1 week at 30 degrees Celsius. Spores were stored refrigerated and were used for the inoculation of liquid media.

An A. niger strain containing multiple copies of the expression cassette was used for generation of sample material by cultivation of the strain in shake flask cultures. A useful method for cultivation of A. niger strains and separation of the mycelium from the culture broth is described in WO 98/46772. Cultivation medium was in CSM-MES (150 g maltose, 60 g Soytone (Difco), 15 g (NH₄)₂SO₄, 1 g NaH₂PO₄H₂PO₄H₂O, 1 g MgSO₄7H₂O, 1 g L-arginine, 80 mg Tween-80, 20 g MES pH6.2 per liter medium). 5 ml samples were taken on day 4-8 of the fermentation, centrifuged for 10 min at 5000 rpm in a Hereaus labofuge RF and supernatants were stored at −20° C. until further analyses.

It became clear that transformants containing the pGBFINPAD vector produced a protein of apparent molecular weight of approximately 60 kDa when analyzed with SDS-PAGE. Since this is slightly smaller than the molecular weight that is predicted from the protein sequence, we presume that after removal of the signal sequence no extensive glycosylation takes place when Fusarium PAD is produced in Aspergillus niger.

Selected strains can be used for isolation and purification of a larger amount of PAD, when fermentation and down-stream processing is scaled up. This enzyme can than be used for further analysis, and for the use in diverse industrial applications.

### Example 4

### Purification of the Over Expressed, Putative PAD From an A. Niger Supernatant

A. niger incorporating plasmid pGBFINPAD was grown on a 10 liter scale using a growth medium incorporating per liter maltose.H₂O: 40 g; Soytone (Difco); 30 g; (NH₄)₂SO₄: 15 g, NaH₂PO₄.H₂O: 1 g; MgSO₄.7H₂O: 1, L-arginine: 1 g, Tween-80: 0.08 g, Na-citrate: 70 g. The pH was adjusted to 6.2. After 6 days of growth at 30 degrees C., cells were killed off by adding 3.5 g/l of sodium benzoate and prolonging incubation for another 6 hours. Then 10 g/l of CaCl2 and 45 g/l filter aid (Dicalite B F; Gent, Belgium) were added to the broth. Mycelium was removed by an initial cloth filtration, followed by filtration through Z-2000 and Z-200 filters (Pall). Finally sterile filtration was carried out using a 0.22 micrometer GP Express PLUS membrane (Millipore). Ultrafiltration was carried out using a Pellicon system (Millipore).

After changing the buffer conditions to 25 mM Na-citrate, pH 5.0 (Buffer A), the solution was applied to a SP-Sepharose XK column (GE Health Care; Diegem, Belgium) equilibrated with the same buffer. The column was eluted in a linear gradient from buffer A to buffer A+1M NaCl. Fractions obtained were subjected to SDS-PAGE and stained. Fractions showing a clear band with an apparent MW of 60 kDa were pooled, The resulting enzyme solution was stabilized by adding glycerol (50% w/w final concentration), CaCl2 (0.02% w/w), and sodium benzoate (0.1% w/w). To estimate the PAD protein concentration in this final preparation, different quantities of the final preparation together with different quantities of a BSA solution (Fraction V, Sigma) were subjected to SDS-PAGE and stained with Simply Blue Safe Stain, According to the results obtained (cf FIG. 4), the PAD concentration in the final preparation is 3.8 mg protein/ml liquid.

### Example 5

### The Overexpressed and Purified Putative PAD can Convert Peptide Bound Arginine into Citrulline

To confirm the nature of the enzyme isolated as a PAD, the synthetic peptide QPRPFPFPRPR (Pepscan, Almere, The Netherlands) was incubated with the chromatographically purified enzyme and the resulting products were analyzed using LC/MS. Aim of the LC/MS analysis was to confirm the conversion of arginine residues into citrulline residues. To that end various quantities of the purified enzyme were incubated with the synthetic peptide (10 mM) at pH 6.5 and 50 degrees C. Samples were withdrawn from the incubation mixture after 0, 1 and 4 hours of incubation. All samples were heated for 10 minutes at 95 degrees C. to inactivate any residual enzyme activity and subsequently centrifuged. The reference sample (0 hours of incubation) was heat-treated and centrifugated immediately after adding the peptide to the incubation mixture. The clear supernatants were subjected to LC/MS analysis under conditions specified in the Materials & Methods section.

For optimizing the MS a 5 μg/ml solution of QPRPFPFPRPR was used. The undeca-peptide was characterized in ESl/pos mode by (major) m/z 697.8=[M+2H]²⁺ and (minor) m/z 465.6=[M+3H]³⁺. For the determination of the retention time of QPRPFPFPRPR, LC/MS was performed. The protonated mass of arginine (R) is 175, after conversion to citrulline (R═O) the protonated mass is increased 1 Da to 176. Due to the tact that the peptides are not characterized by [M+H]⁺ but by the doubly charged [M+2H]²⁺, the mass difference will be characterized by 0.5 Da difference per converted arginine, as the mass axis definition is mass to charge ratio (m/z). The mass chromatogram of the 0 hours incubated sample shows a large peak representing peptide QPRPFPFPRPR (m/z 697.8) at 9.72 minutes. However, a small peak eluting at 11.36 minutes indicated that even in this sample some arginine residues have been converted to citrulline. In the mass chromatogram of the 1 hour incubated sample 3 peaks were apparent. Besides the peaks present after 0 hours incubation, peaks were detected at 13.36 minutes (QPRPFPFPRPR with 2 R's converted to R═O) and at 15.94 minutes (QPRPFPFPRPR with 3 R's converted to R═O). in the sample incubated for 4 hours only the two latter peaks are visible. These data form a strong indication that indeed the enzyme fraction isolated represents an enzyme with PAD activity. Quite surprising is that the data obtained also allowed us to conclude that the PAD starts at the N-terminus of the peptide as arginine residues towards the N-terminus of the peptide are deiminated first. Arginine residues towards the C-terminal end of the peptide follow later.

### Example 6

### Protein-Bound Arginine, Peptide-Bound Arginine and Free Arginine Form Suitable Substrate for the Over-Expressed Fusarium PAD

The availability of larger amounts of the PAD allowed us to investigate the arginine to citrulline conversion by classical amino acid analysis. To that end we had to establish the position of citrulline in the chromatogram used to quantitate the levels of the free amino acids present. Because it was anticipated that the acid hydrolysis used in amino acid analysis would degrade citrulline into ornithine, pure citrulline and ornithine (both from Sigma) were derivatised and subjected to chromatography according to the PicoTag method. A test run demonstrated that both derivatised compounds could be traced back among the other derivatised amino acids as individual peaks in the chromatogram.

In testing the new enzyme, 10% (w/w) solutions of three different substrates, i.e. sodium caseinate, casein hydrolysate and free arginine were incubated with 0, 5, 50 and 500 microliters of the over-expressed and purified PAD (cf. Example 4). Incubation was carried out for 3 hours at pH 6.5 and 45 degrees C. The sodium caseinate was obtained from DMV International, Veghel, The Netherlands, the casein hydrolysate was prepared by a digestion of the sodium caseinate with subtilisin and a proline specific endoprotease (Edens et al, JAFC 53 (20), 7950-7957, 2005). L-arginine was obtained from Sigma.

After incubation, the various mixtures were analysed by amino acid analysis. As can be concluded from the data presented in Table 1, in all incubations containing a substantial amount of the newly identified PAD, significant amounts of arginine were converted into citrulline. As expected the chromatograms also indicated the presence of ornithine. These findings imply that the Fusarium PAD can convert protein-bound and peptide-bound arginine. Quite surprisingly, also free arginine is accepted as a substrate. According to the results, the smaller the substrate, the more effective the enzyme is in in converting arginine into citrulline. The latter observation seems in contrast with the data published for the Porphyromonas gingivalis enzyme which reportedly exhibis a preference for peptidyl arginine substrates rather than free arginine (McGraw et al., Infection and immunity, July 1999, 3248-3256).

To confirm that the presence of ornithine is the result of the acid hydrolysis of cirtrulline, the experiment with the free arginine as the substrate was repeated but this time the acid hydrolysis was omitted. As shown in Table 2, in this case the amounts of ornithine formed are negligible demonstrating that the ornithine present is not a product formed by the enzyme.

TABLE 1
Conversion of bound and free arginine into citrulline

| | Microliter | | | |
|---|---|---|---|---|
| Substrate used | PAD | arginine | citrulline | ornithine |
| Caseinate | 0 | 100.0% | | |
| | 5 | 97.8% | 0.8% | 1.3% |
| | 50 | 86.5% | 4.7% | 8.8% |
| | 500 | 70.2% | 10.5% | 19.3% |
| Hydrolysate | 0 | 100.0% | | |
| | 5 | 97.2% | 0.9% | 1.9% |
| | 50 | 86.1% | 5.0% | 8.9% |
| | 500 | 60.4% | 15.1% | 24.5% |
| Arginine | 0 | 99.1% | | |
| | 5 | 99.3% | | |
| | 50 | 80.0% | 7.2% | |
| | 500 | 14.7% | 32.3% | 53.0% |

TABLE 2
Omitting acid hydrolysis prevents ornithine formation

| | Microliter | | | |
|---|---|---|---|---|
| Substrate used | PAD | arginine | citrulline | ornithine |
| Arginine | 0 | 100.0% | | |
| | 5 | 100.0% | | |
| | 50 | 82.8% | 17.2% | |
| | 500 | 18.8% | 80.5% | 0.7% |

### Example 7

### The pH and Temperature Optima of the Over-Expressed PAD

In a set of experiments very similar to the one described in Example 6, the pH and the temperature optima of the Fusarium PAD were determined. According to these results, the overexpressed enzyme has its pH optimum around 8.0 and its temperature optimum between 40 and 50 degrees C.

### Example 8

### Calcium Dependency of the Over-Expressed PAD

According to the literature the eukaryotic petidylarginine deiminases represent a family of Ca 2+-dependent enzymes. To test if the Fusarium PAD also requires Ca 2+, an incubation was carried out in the presence of EDTA. To that end sodium caseinate was first desalted using a Desalt Spin Column (Pierce, Rockford, Ill.) and a 5% caseinate solution was then incubated with for 4 hours at 45 degrees C. and pH 7.2 with PAD in the presence and absence of 10 mM (final concentration) of EDTA. According to the amino acid analyses carried out, both incubations showed the same reduction (approx 20%) of free arginine in the acid hydrolysed caseinate indicating that the Fusarium PAD is Ca2+ independent.

### Example 9

Soft Drink With 30% Juice
Typical serving: 240 ml
Active ingredients:
Casein hydrolysate (50% of arginine is converted into citrulline) and maltodextrin as a carbohydrate source are incorporated in this food item:
Casein hydrolysate; 1.5-15 g/per serving
Maltodextrin: 3-30 g/per serving

### I. A Soft Drink Compound is Prepared From the Following Ingredients:

### Juice Concentrates and Water Soluble Flavors

| | [g] |
|---|---|
| 1.1 Orange concentrate | |
| 60.3° Brix, 5.15% acidity | 657.99 |
| Lemon concentrate | |
| 43.5° Brix, 32.7% acidity | 95.96 |
| Orange flavor, water soluble | 13.43 |
| Apricot flavor, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavors | |
| Orange flavor, oil soluble | 0.34 |
| Orange oil distilled | 0.34 |

### 1.6 Active Ingredients

Active ingredients (this means the active ingredient mentioned above) protein hydrolysate and maltodextrin in the concentrations mentioned above,
Fruit juice concentrates and water soluble flavors are mixed without incorporation of air. The color is dissolved in deionized water. Ascorbic acid and citric acid is dissolved in water. Sodium benzoate is dissolved in water. The pectin is added under stirring and dissolved while boiling. The solution is cooled down. Orange oil and oil soluble flavors are premixed. The active ingredients as mentioned under 1.6 are dry mixed and then stirred preferably into the fruit juice concentrate mixture (1.1).
In order to prepare the soft drink compound all parts 3.1.1 to 3.1.6 are mixed together before homogenizing using a Turrax and then a high-pressure homogenizer (p₁=200 bar, p₂ =50 bar).

### II. A Bottling Syrup is Prepared From the Following Ingredients:

| | [g] |
|---|---|
| Softdrink compound | 74.50 |
| Water | 50.00 |
| Sugar syrup 60° Brix | 150.00 |

The ingredients of the bottling syrup are mixed together. The bottling syrup is diluted with water to 1 l of ready to drink beverage.

### Variations:

Instead of using sodium benzoate, the beverage may be pasteurized. The beverage may also be carbonized.

1. A protein, peptide or protein hydrolysate wherein the molar ratio of citrulline and arginine residues, being part of protein or peptide, is at least 0.15.

2. The modified protein, peptide or protein hydrolysate of claim 1 which is a vegatale protein, skim milk protein, milk protein, whet protein, casein protein, gelatin protein, egg protein, microbial protein or a hydrolysate thereof.

3. A protein hydrolysate according to claim 1 wherein the average peptide length is from 3 to 9 amino acids.

4. A protein hydrolysate according to claim 1 having a DH of between 5 and 50.

5. Use of a protein, peptide or protein hydrolysate according to claim 1 in a food, a feed or a nutraceutical, such as a dietary supplement or a medicament and in the preparation of a food, a feed or a nutraceutical such as a dietary supplement or medicament

6. A method of enzymatically producing a protein, peptide or protein hydrolysate wherein at least 15% of the arginine residues which were originally present in the protein, peptide or protein hydrolysate is transformed into a citrulline residue in which method the protein, peptide or protein hydrolysate substrate is incubated with a protein arginine deiminase.

7. A foodstuff comprising a protein, peptide or protein hydrolysate according to claim 1.

8. A foodstuff according to claim 7 which is an infant formula or a clinical food.

9. An isolated polypeptide which has protein arginine deiminase activity, selected from the group consisting of:
(a) a polypeptide which has an amino acid sequence which has at least 30% amino acid sequence identity with amino acids 1 to 640 of SEQ ID NO: 6, 8, 9, 10, 13 or 14;
(b) a polypeptide which is encoded by a polynucleotide which hybridizes under low stringency conditions with (i) the nucleic acid sequence of SEQ ID NO: 3 or a fragment thereof which is at least 90% identical over 200 nucleotides, or (ii) a nucleic acid sequence complementary to the nucleic acid sequence of SEQ ID NO: 3.

10. The polypeptide of claim 9 which has an amino acid sequence which has at least 90% identity with amino acids 1 to 640 of SEQ ID No: 6.

11. The polypeptide of claim 9, comprising the amino acid sequence of SEQ ID NO: 6.

12. The polypeptide of claim 9, which is encoded by a polynucleotide that hybridizes under high stringency conditions, with (i) the nucleic acid sequence of SEQ ID NO: 3 or a fragment thereof, or (ii) a nucleic acid sequence complementary to the nucleic acid sequence of SEQ ID NO: 3.

13. The polypeptide of claim 9, which is obtained from a fungus.

14. An isolated polynucleotide comprising a nucleic acid sequence which encodes the polypeptide of claim 9, or which hybridizes with SEQ ID NO: 3 under high stringency conditions.

15. A polynucleotide which encodes a polypeptide which has protein arginine deiminase activity said polynucleotide comprises
(a) a polynucleotide sequence which encodes amino acid SEQ ID NO: 11, and
(b) a polynucleotide sequence which encodes a pre-protein signal sequence whereby the encoded pre-protein signal sequence is located at the amino terminus of the encoded pre-polypeptide and is preferably 15 to 30 amino acids in length.

16. A nucleic acid construct comprising the polynucleotide of claim 14 operably linked to one or more control sequences that direct the production of the polypeptide in a suitable expression host.

17. A recombinant expression vector comprising the nucleic acid construct of claim 16,

18. A recombinant host cell comprising the nucleic acid construct of claim 16.

19. A method for producing a protein arginine deiminase which comprises cultivating a strain or recombinant host which is capable of secreting protein arginine deiminase and recovering the protein arginine deiminase.

20. A method for producing the polypeptide of claim 9 comprising cultivating a strain/recombinant host cell, to produce a supernatant and/or cells comprising the polypeptide; and recovering the polypeptide.

21. A polypeptide produced by the method of claim 19.

22. A method for producing the polypeptide of claims 9 comprising cultivating a host cell comprising a nucleic acid construct comprising a polynucleotide encoding the polypeptide under conditions suitable for production of the polypeptide; and recovering the polypeptide.

23. A polypeptide produced by the method of claim 20.

24. A DNA molecule encoding an protein arginine deiminase according to claim 9.
